SEQUENCE LISTING (55 sequences)

SEQ ID NO: 1 - DNA, 14 bp, artificial sequence
  3′ primer
ttttttttttttna

SEQ ID NO: 2 - DNA, 14 bp, artificial sequence
  3′ primer
ttttttttttttnc

SEQ ID NO: 3 - DNA, 14 bp, artificial sequence
  3′ primer
ttttttttttttng

SEQ ID NO: 4 - DNA, 14 bp, artificial sequence
  3′ primer
ttttttttttttnt

SEQ ID NO: 5 - DNA, 10 bp, artificial sequence
  3′ primer
caggcccttc

SEQ ID NO: 6 - DNA, 10 bp, artificial sequence
  3′ primer
tgccgagctg

SEQ ID NO: 7 - DNA, 10 bp, artificial sequence
  3′ primer
agtcagccac

SEQ ID NO: 8 - DNA, 10 bp, artificial sequence
  3′ primer
aatcgggctg

SEQ ID NO: 9 - DNA, 10 bp, artificial sequence
  3′ primer
aggggtcttg

SEQ ID NO: 10 - DNA, 10 bp, artificial sequence
  3′ primer
ggtccctgac

SEQ ID NO: 11 - DNA, 10 bp, artificial sequence
  3′ primer
gaaacgggtg

SEQ ID NO: 12 - DNA, 10 bp, artificial sequence
  3′ primer
gtgacgtagg

SEQ ID NO: 13 - DNA, 10 bp, artificial sequence
  3′ primer
gggtaacgcc

SEQ ID NO: 14 - DNA, 10 bp, artificial sequence
  3′ primer
gtgatcgcag

SEQ ID NO: 15 - DNA, 10 bp, artificial sequence
  3′ primer
caatcgccgt

SEQ ID NO: 16 - DNA, 10 bp, artificial sequence
  3′ primer
tcggcgatag

SEQ ID NO: 17 - DNA, 10 bp, artificial sequence
  3′ primer
cagcacccac

SEQ ID NO: 18 - DNA, 10 bp, artificial sequence
  3′ primer
tctgtgctgg

SEQ ID NO: 19 - DNA, 10 bp, artificial sequence
  3′ primer
ttccgaaccc

SEQ ID NO: 20 - DNA, 10 bp, artificial sequence
  3′ primer
agccagcgaa

SEQ ID NO: 21 - DNA, 10 bp, artificial sequence
  3′ primer
gaccgcttgt

SEQ ID NO: 22 - DNA, 10 bp, artificial sequence
  3′ primer
aggtgaccgt

SEQ ID NO: 23 - DNA, 10 bp, artificial sequence
  3′ primer
caaacgtcgg

SEQ ID NO: 24 - DNA, 10 bp, artificial sequence
  3′ primer
gttgcgatcc

SEQ ID NO: 25 - DNA, 359 bp, Mus musculus
tctgtgctggccaggatggacaggaagatgagtttcataatcacatggtctccaaccctgacagctcattctcccaaggtgactacacggtggccaaagaggagcggacacctgcctgaggtgcaaggactgagccacttcacctctgcatgcagttctgggtgcggcagctgtctatgaagatggcgccacccagcagccagcaggctcccaagggcatctttgttctccctagtgtttcaagtgtatttgtgagcattgctgtaaagtttctcccactacccacattgcttgtactgtatgtttctctactgtatggcattaaagtttacaagcacatagctgccaaaaaaaaaaaa

SEQ ID NO: 26 - DNA, 2450 bp, Mus musculus
gaagctcggctgagagaggcccgggtcagtccccacaccatgccctgtttgcgctccgggccagagtgcgcctgagcggttcgggcctcggtatccccgcgggtcttgcgccgccgcctctccgcgatgcaggcgcgcgcgctgctccccgccacgctggccattctggccacgctggctgtgttggctctggcccgggagcccccagcggctccgtgtcctgcgcgctgcgacgtgtcgcgctgtccgagccctcgctgccctgggggctatgtgcctgacctctgcaactgctgcctggtgtgcgctgccagcgagggcgagccctgcggccgccccctggactctccgtgcggggacagtctggagtgcgtgcgcggcgtgtgccgctgccgttggacccacactgtgtgtggcacagacgggcatacttatgccgacgtgtgcgcgctgcaggccgccagccgtcgtgcgttgcaggtctccgggactccagtgcgccagctgcagaagggtgcctgtccctctggtctccaccagctgaccagtccgcggtacaagttcaacttcatcgccgatgtggtggagaagattgcgccagctgtggtccacatagagctctttctgagacaccccctgtttggccggaatgtgccgctgtccagtggctcgggcttcatcatgtcagaagccggtttgatcgtcaccaacgcccacgtggtctccagctccagcactgcctccggccggcagcagctgaaggtgcagctgcagaatggggatgcctatgaggccaccatccaggacatcgacaagaagtcggacattgccacgattgtaatccaccccaagaaaaagctccctgtgttgctgctgggtcactcagcagacctgcggcctggcgagttcgtggtggccatcggcagcccctttgccctgcagaacaccgtgacaacgggcattgtcagcactgcccagcgggatggcaaggagctgggtctccgggactcagacatggactatatccagaccgatgccatcatcaattacgggaactcaggaggacccctggtgaacctggatggcgaggtcatcggcatcaacacgctcaaggttgcagctggcatctcctttgccatcccctcagatcgcatcacacgcttcctctctgagttccaaaacaagcatgtgaaagactggaagaagcgcttcattggcatccggatgcggaccatcacgccaagtttggtggaggaactgaaggccgccaacccagactttccagcggtcagcagtggaatatatgttcaagaggtggttcccaattcaccttctcagagaggaggcatccaagatggcgacatcatcgtcaaagtcaatggccgccccctggcggattccagcgagctgcaggaggcagtcctgaacgagtcttcactcctgctggaggtgcggcgaggcaatgatgatctcctcttcagcatcatccctgaggtggtcatgtgaggctactctcatccagtgccatgccaaagcctacagaaggtggggttccggccttcatgaaatcaggacaaacggctgctgtggtcctcagcaggatcaacagtctcctctctgggtccagcgctgagtccaaggctggatctaaccaggggtccggatctcagccttgacccttaatttcagctccagtagaggaagcacagcgtcctttggaccagatgctcctgatgttaccgtctgagttctctaggcctagaagctcttagaaacctccctggaagtctgcccttcccccacccccaccccagctttctgcctctgccctcaggaaggcccacccggctcccatcccacctcttctcccttgtatcccagtgcctcaacctctccctgttacaggcactttcctgacactaccaggcttccatctgcctcagcacaccccacccccatggtaagacaggggctgcttgccctaccacccggtatccctggagggcaggccctgtagctgtcccctggagaagccagggtcctgacctggagcaggttaacatccctcactgctgagctgagccctgtgctggccaggatggacaggaagatgagtttcataatcacgtggtctccaaccctgacagctcattctcccaaggtgactacacggtggccaaagaggagcggacacctgcctgaggtgcaaggactgagccacttcacctctgcatgcagttctgggtgcggcagctgtctgtgaagatggcgccacccagcagccagcaggctcccaagggcatctttgttctccctagtgtttcaagtgtatttgtgagcattgctgtaaagtttctcccactacccacattgcttgtactgtatgtttctctactgtatggcattaaagtttacaagcacatagctgtcaaccagaaaaaaaaaattcc

SEQ ID NO: 27 - protein, 459 aa, Mus musculus
MQARALLPATLAILATLAVLALAREPPAAPCPARCDVSRCPSPRCPGGYVPDLCNCCLVCAASEGEPCGRPLDSPCGDSLECVRGVCRCRWTHTVCGTDGHTYADVCALQAASRRALQVSGTPVRQLQKGACPSGLHQLTSPRYKFNFIADVVEKIAPAVVHIELFLRHPLFGRNVPLSSGSGFIMSEAGLIVTNAHVVSSSSTASGRQQLKVQLQNGDAYEATIQDIDKKSDIATIVIHPKKKLPVLLLGHSADLRPGEFVVAIGSPFALQNTVTTGIVSTAQRDGKELGLRDSDMDYIQTDAIINYGNSGGPLVNLDGEVIGINTLKVAAGISFAIPSDRITRFLSEFQNKHVKDWKKRFIGIRMRTITPSLVEELKAANPDFPAVSSGIYVQEVVPNSPSQRGGIQDGDIIVKVNGRPLADSSELQEAVLNESSLLLEVRRGNDDLLFSIIPEVVM

SEQ ID NO: 28 - protein, 7 aa, artificial sequence
  Bacterial HtrA activwe site motif
GNSGGAL

SEQ ID NO: 29 - protein, 7 aa, artificial sequence
  Mammalian HtrA active site motif
GNSGGPL

SEQ ID NO: 30 - protein, 5 aa, artificial sequence
  HtrA second active site motif
TNAHV

SEQ ID NO: 31 - DNA, 2543 bp, Homo sapiens
gtgcgctccctgcgccctggggatgcccctgccgccctgacgcccgccagcctgagccaccggcgcatgtgaccgcgcgtccgccccagtcccatccgtaggcgcccggcgcccggccccgcagcggcctcgttgtccccgccggcccccgcccggtctcccgcgctgccacccgccgccggccctgccgccatgcaggcgcgagcgctgctcctggccgcgttggccgcgctggcgctggcccgggagccccctgcggcgccgtgtcccgcgcgctgcgacgtgtcgcggtgtcccagcccccgctgccccggcggctacgtgcccgacctctgcaactgctgcctggtgtgcgccgccagcgagggcgagccctgtggcggccctctggactcgccttgcggcgagagcctggagtgcgtgcgcggcctatgccgctgccgctggtcgcacgccgtgtgtggcaccgacgggcacacctatgccaacgtgtgcgcgctgcaggcggccagccgccgcgcgctgcagctctccgggacgcccgtgcgccagctgcagaagggcgcctgcccgttgggtctccaccagctgagcagcccgcgctacaagttcaacttcattgctgacgtggtggagaagatcgcaccagccgtggtccacatagagctcttcctgagacacccgctgtttggccgcaacgtgcccctgtccagcggttctggcttcatcatgtcagaggccggcctgatcatcaccaatgcccacgtggtgtccagcaacagtgctgccccgggcaggcagcagctcaaggtgcagctacagaatggggactcctatgaggccaccatcaaagacatcgacaagaagtcggacattgccaccatcaagatccatcccaagaaaaagctccctgtgttgttgctgggtcactcggccgacctgcggcctggggagtttgtggtggccatcggcagtcccttcgccctacagaacacagtgacaacgggcatcgtcagcactgcccagcgggagggcagggagctgggcctccgggactccgacatggactacatccagacggatgccatcatcaactacgggaactccgggggaccactggtgaacctggatggcgaggtcattggcatcaacacgctcaaggtcacggctggcatctcctttgccatcccctcagaccgcatcacacggttcctcacagagttccaagacaagcagatcaaagactggaagaagcgcttcatcggcatacggatgcggacgatcacaccaagcctggtggatgagctgaaggccagcaacccggacttcccagaggtcagcagtggaatttatgtgcaagaggttgcgccgaattcaccttctcagagaggcggcatccaagatggtgacatcatcgtcaaggtcaacgggcgtcctctagtggactcgagtgagctgcaggaggccgtgctgaccgagtctcctctcctactggaggtgcggcgggggaacgacgacctcctcttcagcatcgcacctgaggtggtcatgtgaggggcgcattcctccagcgccaagcgtcagagcctgcagacaacggagggcagcgcccccccgagatcaggacgaaggaccaccgtcggtcctcagcagggcggcagcctcctcctggctgtccggggcagagcggaggctgggcttggccaggggcccgaatttccgcctggggagtgttggatccacatcccggtgccggggagggaagcccaacatccccttgtacagatgatcctgaaagtcacttccaagttctccggatattcacaaaactgccttccatggaggtcccctcctctcctagcttcccgcctctgcccctgtgaacacccatctgcagtatcccctgctcctgcccctcctactgcaggtctgggctgccaagcttcttcccccctgacaaacgcccacctgacctgaggccccagcttccctctgccctaggacttaccaagctgtagggccagggctgctgcctgccagcctggggtccctggaggacaggtcacatctgatccctttggggtgcgggggtggggtccagcccagagcaggcactgagtgaatgccccctggctgcggagctgagccccgccctgccatgaggttttcctccccaggcaggcaggaggccgcggggagcacgtggaaagttggctgctgcctggggaagcttctcctccccaaggcggccatggggcagcctgcagaggacagtggacgtggagctgcggggtgtgaggactgagccggcttccccttcccacgcagctctgggatgcagcagccgctcgcatggaagtgccgcccagaggcatgcaggctgctgggcaccaccccctcatccagggaacgagtgtgtctcaaggggcatttgtgagctttgctgtaaatggattcccagtgttgcttgtactgtatgtttctctactgtatggaaaataaagtttacaagcacacggttctcaaaaaaaaaaaaaaaaaaa

SEQ ID NO: 32 - DNA, 1953 bp, Homo sapiens
ccagtcccatccgtaggcgcccggcgcccggccccgcagcggcctcgttgtccccgccggcccccgcccggtctcccgcgctgccacccgccgccggccctgccgccatgcaggcgcgagcgctgctcctggccgcgttggccgcgctggcgctggcccgggagccccctgcggcgccgtgtcccgcgcgctgcgacgtgtcgcggtgtcccagcccccgctgccccggcggctacgtgcccgacctctgcaactgctgcctggtgtgcgccgccagcgagggcgagccctgtggcggccctctggactcgccttgcggcgagagcctggagtgcgtgcgcggcctatgccgctgccgctggtcgcacgccgtgtgtggcaccgacgggcacacctatgccaacgtgtgcgcgctgcaggcggccagccgccgcgcgctgcagctctccgggacgcccgtgcgccagctgcagaagggcgcctgcccgttgggtctccaccagctgagcagcccgcgctacaagttcaacttcattgctgacgtggtggagaagatcgcaccagccgtggtccacatagagctcttcctgagacacccgctgtttggccgcaacgtgcccctgtccagcggttctggcttcatcatgtcagaggccggcctgatcatcaccaatgcccacgtggtgtccagcaacagtgctgccccgggcaggcagcagctcaaggtgcagctacagaatggggactcctatgaggccaccatcaaagacatcgacaagaagtcggacattgccaccatcaagatccatcccaagaaaaagctccctgtgttgttgctgggtcactcggccgacctgcggcctggggagtttgtggtggccatcggcagtcccttcgccctacagaacacagtgacaacgggcatcgtcagcactgcccagcgggagggcagggagctgggcctccgggactccgacatggactacatccagacggatgccatcatcaactacgggaactccgggggaccactggtgaacctggatggcgaggtcattggcatcaacacgctcaaggtcacggctggcatctcctttgccatcccctcagaccgcatcacacggttcctcacagagttccaagacaagcagatcaaagccccctcactggcagttcattgagagcagggggcttcctcacgtttccccctcctccatgaccccgtcagccaagcacatggaccccagtgcagccaaggctggtgccatgagggctggtcacatgaagagctgctgttgaggatgccgccattgttcttctgtgtccattatgggaagacaatctggagccaggcagagcctgtctttcccaaagaagctgaagtcttcttctcttgaacagtggggaccatctaatctcttgagcccttttcctgttggcttctaggaagctcagagctagattcaggggtgcacccagacctgtcctagcatgctcctttccctaatgaccgagtctttcctgttgaattatcccattctccatgggtgcctttgactttggcctccttactggaaattagcggagctgctgtttgcacacactgagctgtgaggtggctttccttggaagtggatgatagtgtcctcttcccttcttgcctctctctttctcctgagacaggatccccctggggcctaggtttgctcctttgttgtacaggggctgtcccagttagtgctgacctcatcccagaaccccctgggaaatatcccctgtcctcagagctgtgtcccctccccaaggacagtgcagactaactgaggagcctgataaaccttagctgcatggcacacttgcaattttaaaatccttctgaagttgactggtgtttgtacttgcttctcttttttatttaataaaatccaatgatccaaaaaaaaaaaaaaaaaa

SEQ ID NO: 33 - protein, 453 aa, Homo sapiens
MQARALLLAALAALALAREPPAAPCPARCDVSRCPSPRCPGGYVPDLCNCCLVCAASEGEPCGGPLDSPCGESLECVRGLCRCRWSHAVCGTDGHTYANVCALQAASRRALQLSGTPVRQLQKGACPLGLHQLSSPRYKFNFIADVVEKIAPAVVHIELFLRHPLFGRNVPLSSGSGFIMSEAGLIITNAHVVSSNSAAPGRQQLKVQLQNGDSYEATIKDIDKKSDIATIKIHPKKKLPVLLLGHSADLRPGEFVVAIGSPFALQNTVTTGIVSTAQREGRELGLRDSDMDYIQTDAIINYGNSGGPLVNLDGEVIGINTLKVTAGISFAIPSDRITRFLTEFQDKQIKDWKKRFIGIRMRTITPSLVDELKASNPDFPEVSSGIYVQEVAPNSPSQRGGIQDGDIIVKVNGRPLVDSSELQEAVLTESPLLLEVRRGNDDLLFSIAPEVVM

SEQ ID NO: 34 - protein, 357 aa, Homo sapiens
MQARALLLAALAALALAREPPAAPCPARCDVSRCPSPRCPGGYVPDLCNCCLVCAASEGEPCGGPLDSPCGESLECVRGLCRCRWSHAVCGTDGHTYANVCALQAASRRALQLSGTPVRQLQKGACPLGLHQLSSPRYKFNFIADVVEKIAPAVVHIELFLRHPLFGRNVPLSSGSGFIMSEAGLIITNAHVVSSNSAAPGRQQLKVQLQNGDSYEATIKDIDKKSDIATIKIHPKKKLPVLLLGHSADLRPGEFVVAIGSPFALQNTVTTGIVSTAQREGRELGLRDSDMDYIQTDAIINYGNSGGPLVNLDGEVIGINTLKVTAGISFAIPSDRITRFLTEFQDKQIKAPSLAVH

SEQ ID NO: 35 - DNA, 17 bp, artificial sequence
  Forward primer for splice site
ggcatcaacacgctcaa

SEQ ID NO: 36 - DNA, 39 bp, artificial sequence
  Backward primer for splice site
gaccacgcgtatcgatgtcgacttttttttttttttttv

SEQ ID NO: 37 - DNA, 476 bp, artificial sequence
  Probe for short isoform of mouse uterine protease
ccatgaagaactgcaaccgaggagcctcgttctgttccaagtggccctatatgaagatgacaggagcaggcagagcctgtcccttccaggaatccgagacaccttctggtgaatagtgggaactagctgccttttctcttggccggtaggaagctcagaactagaccagggttcctagaccattggtagccttggctctttgtctagtggccagggctttccagtttagcttgtttatggggtcggaacaccacccacatacactggcctatgggtgattactgtgctggaaatgggccagcggccttttgtcccctagctgtctcatcttttctcagacaagaagtccccggggcaggatctgctcctctgtggcagagcaactatcctagtcacagtgacctggtcactcagcctgggctctgcggaaatgctcacacccatcccagagttatgttatcacccaaggacagtgc

SEQ ID NO: 38 - DNA, 1897 bp, Mus musculus
gaagctcggctgagagaggcccgggtcagtccccacaccatgccctgtttgcgctccgggccagagtgcgcctgagcggttcgggcctcggtatccccgcgggtcttgcgccgccgcctctccgcgatgcaggcgcgcgcgctgctccccgccacgctggccattctggccacgctggctgtgttggctctggcccgggagcccccagcggctccgtgtcctgcgcgctgcgacgtgtcgcgctgtccgagccctcgctgccctgggggctatgtgcctgacctctgcaactgctgcctggtgtgcgctgccagcgagggcgagccctgcggccgccccctggactctccgtgcggggacagtctggagtgcgtgcgcggcgtgtgccgctgccgttggacccacactgtgtgtggcacagacgggcatacttatgccgacgtgtgcgcgctgcaggccgccagccgtcgtgcgttgcaggtctccgggactccagtgcgccagctgcagaagggtgcctgtccctctggtctccaccagctgaccagtccgcggtacaagttcaacttcatcgccgatgtggtggagaagattgcgccagctgtggtccacatagagctctttctgagacaccccctgtttggccggaatgtgccgctgtccagtggctcgggcttcatcatgtcagaagccggtttgatcgtcaccaacgcccacgtggtctccagctccagcactgcctccggccggcagcagctgaaggtgcagctgcagaatggggatgcctatgaggccaccatccaggacatcgacaagaagtcggacattgccacgattgtaatccaccccaagaaaaagctccctgtgttgctgctgggtcactcagcagacctgcggcctggcgagttcgtggtggccatcggcagcccctttgccctgcagaacaccgtgacaacgggcattgtcagcactgcccagcgggatggcaaggagctgggtctccgggactcagacatggactatatccagaccgatgccatcatcaattacgggaactcaggaggacccctggtgaacctggatggcgaggtcatcggcatcaacacgctcaaggttgcagctggcatctcctttgccatcccctcagatcgcatcacacgcttcctctctgagttccaaaacaagcatgtgaaagccctctcaccagcactgcactgagagcaggggccttcctcctgcttgccccctcctttgcggccctgccagccacacacaaggaccccagtacagccaagactggtcccatgaagaactgcaaccgaggagcctcgttctgttccaagtggccctatatgaagatgacaggagcaggcagagcctgtcccttccaggaatccgagacaccttctggtgaatagtgggaactagctgccttttctcttggccggtaggaagctcagaactagaccagggttcctagaccattggtagccttggctctttgtctagtggccagggctttccagtttagcttgtttatggggtcggaacaccacccacatacactggcctatgggtgattactgtgctggaaatgggccagcggccttttgtcccctagctgtctcatcttttctcagacaagaagtccccggggcaggatctgctcctctgtggcagagcaactatcctagtcacagtgacctggtcactcagcctgggctctgcggaaatgctcacacccatcccagagttatgttatcacccaaggacagtgcttacctactacaagagggtctgacgaggcttagctaagtggggtccattgacttaaagtccttctgaaatttgtgcttatttatgcttttccatttttaaataaaaacatcagatgatc

SEQ ID NO: 39 - protein, 363 aa, Mus musculus
MQARALLPATLAILATLAVLALAREPPAAPCPARCDVSRCPSPRCPGGYVPDLCNCCLVCAASEGEPCGRPLDSPCGDSLECVRGVCRCRWTHTVCGTDGHTYADVCALQAASRRALQVSGTPVRQLQKGACPSGLHQLTSPRYKFNFIADVVEKIAPAVVHIELFLRHPLFGRNVPLSSGSGFIMSEAGLIVTNAHVVSSSSTASGRQQLKVQLQNGDAYEATIQDIDKKSDIATIVIHPKKKLPVLLLGHSADLRPGEFVVAIGSPFALQNTVTTGIVSTAQRDGKELGLRDSDMDYIQTDAIINYGNSGGPLVNLDGEVIGINTLKVAAGISFAIPSDRITRFLSEFQNKHVKALSPALH

SEQ ID NO: 40 - DNA, 785 bp, artificial sequence
  Probe for common region of short and long isoforms of mouse uteri ne protease
gcggttcgggcctcggtatccccgcgggtcttgcgccgccgcctctccgcgatgcaggcgcgcgcgctgctccccgccacgctggccattctggccacgctggctgtgttggctctggcccgggagcccccagcggctccgtgtcctgcgcgctgcgacgtgtcgcgctgtccgagccctcgctgccctgggggctatgtgcctgacctctgcaactgctgcctggtgtgcgctgccagcgagggcgagccctgcggccgccccctggactctccgtgcggggacagtctggagtgcgtgcgcggcgtgtgccgctgccgttggacccacactgtgtgtggcacagacgggcatacttatgccgacgtgtgcgcgctgcaggccgccagccgtcgtgcgttgcaggtctccgggactccagtgcgccagctgcagaagggtgcctgtccctctggtctccaccagctgaccagtccgcggtacaagttcaacttcatcgccgatgtggtggagaagattgcgccagctgtggtccacatagagctctttctgagacaccccctgtttggccggaatgtgccgctgtccagtggctcgggcttcatcatgtcagaagccggtttgatcgtcaccaacgcccacgtggtctccagctccagcactgcctccggccggcagcagctgaaggtgcagctgcagaatggggatgcctatgaggccaccatccaggacatcgacaagaagtcggacattgccacgattgtaatccaccccaagaaaaagct

SEQ ID NO: 41 - DNA, 384 bp, artificial sequence
  Probe for human HtrA
aaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgagga

SEQ ID NO: 42 - DNA, 20 bp, artificial sequence
  Mouse long isoform upper primer
atgcggacgatcacaccaag

SEQ ID NO: 43 - DNA, 19 bp, artificial sequence
  Mouse long isoform lower primer
cgctgccctccgttgtctg

SEQ ID NO: 44 - DNA, 20 bp, artificial sequence
  Mouse short isoform upper primer
gagggctggtcacatgaaga

SEQ ID NO: 45 - DNA, 18 bp, artificial sequence
  Mouse short isoform lower primer
gctccgctaatttccagt

SEQ ID NO: 46 - DNA, 24 bp, artificial sequence
  HtrA upper primer
aaagccatcaccaagaagaagtat

SEQ ID NO: 47 - DNA, 18 bp, artificial sequence
  HtrA lower primer
tcctcatccgtcatccac

SEQ ID NO: 48 - DNA, 396 bp, Homo sapiens
cggcctgatcatcaccaatgcccacgtggtgtccagcaacagtgctgccccgggcaggcagcagctcaaggtgcagctacagaatggggactcctatgaggccaccatcaaagacatcgacaagaagtcggacattgccaccatcaagatccatcccaagaaaaagctccctgtgttgttgctgggtcactcggccgacctgcggcctggggagtttgtggtggccatcggcagtcccttcgccctacagaacacagtgacaacgggcatcgtcagcactgcccagcgggagggcagggagctgggcctccgggactccgacatggactacatccagacggatgccatcatcaactacgggaactccgggggaccactggtgaacctggatggcga

SEQ ID NO: 49 - protein, 10 aa, artificial sequence
  Antigenic peptide from  mouse uterine protease
PSGLHQLTSP

SEQ ID NO: 50 - protein, 11 aa, artificial sequence
  Antigenic peptide from mouse uterine protease
ALQVSGTPVRQ

SEQ ID NO: 51 - protein, 12 aa, artificial sequence
  Antigenic peptide from region common to both isoforms of mouse uterine protease
GPLVNLDGEVIG

SEQ ID NO: 52 - DNA, 457 bp, Homo sapiens
gcggttctggcttcatcatgtcagaggccggcctgatcatcaccaatgcccacgtggtgtccagcaacagtgctgccccgggcaggcagcagctcaaggtgcagctacagaatggggactcctatgaggccaccatcaaagacatcgacaagaagtcggacattgccaccatcaagatccatcccaagaaaaagctccctgtgttgttgctgggtcactcggccgacctgcggcctggggagtttgtggtggccatcggcagtcccttcgccctacagaacacagtgacaacgggcatcgtcagcactgcccagcgggagggcagggagctgggcctccgggactccgacatggactacatccagacggatgccatcatcaactacgggaactccgggggaccactggtgaacctggatggcgaggtcattggcatcaacacgctcaaggtcacggc

SEQ ID NO: 53 - protein, 10 aa, artificial sequence
  Antigenic peptide from  human uterine protease
PSGLHQLTSP

SEQ ID NO: 54 - protein, 11 aa, artificial sequence
  Antigenic peptide from human uterine protease
ALQLSGTPVRQ

SEQ ID NO: 55 - protein, 12 aa, artificial sequence
  Antigenic peptide from region common to both isoforms of human uterine protease
GPLVNLDGEVIG

(19) United States
(12) Patent Application Publication
(10) Pub. No.: US 2010/0186101 A1
(43) Pub. Date: Jul. 22, 2010

(54) DIAGNOSIS AND TREATMENT OF FERTILITY CONDITIONS USING A SERINE PROTEASE

(76) Inventors: Guiying NIE, Glen Waverley (AU); Lois Adrienne SALAMONSEN, Kew (AU)

Correspondence Address: KLAUBER & JACKSON, 411 HACKENSACK AVENUE, HACKENSACK, NJ, 07601, US

(21) Appl. No.: 12/636,450

(22) Filed: Dec. 11, 2009

Related U.S. Application Data
(63) Continuation-in-part of application No. 11/836,610, filed on Aug. 9, 2007, now abandoned.
Application No. 11/836,610 is a division of application No. 10/485,313, filed on Sep. 22, 2004, now abandoned.
Application No. 10/485,313 is a continuation-in-part of application No. 12/339,203, filed on Dec. 19, 2008.
(60) Provisional application No. 61/015,956, filed on Dec. 21, 2007.

(30) Foreign Application Priority Data
Jul. 30, 2001 (AU) PR6707

(51) Int. Cl. A01K 67/00; G01N 33/573; C07K 16/00

(52) U.S. Cl. 800/9; 435/7.4; 530/387.1; 530/388.1

(57) ABSTRACT

The invention relates to the use of a serine protease, which is specifically expressed in association with embryo implantation and placentation in pregnant uterus in the evaluation of fertility and monitoring of early pregnancy, placental development and function, fetal development, parturition, and conditions such as pre-eclampsia, intrauterine growth restriction, early abortion, abnormal uterine bleeding, endometriosis, and cancers.

### RELATED APPLICATIONS

The present application is a Continuation in Part of copending application Ser. No. 11/836,610, filed Aug. 9, 2007, which is a Divisional of application Ser. No. 10/485,313, filed Sep. 22, 2004, which in turn, is a Nation Stage filing from Application No. PCT/AU02/0101, filed Jul. 30, 2002, and which in turn, claims the priority from Australian application Serial No. PR6707, filed Jul. 30, 2001. This application is also a Continuation In Part of co-pending application Ser. No. 12/339,203, file Dec. 19, 2008, which in turn claims priority from provisional application U.S. Ser. No. 61/015,956, filed Dec. 21, 2007. The disclosures of all of the aforementioned applications are incorporated by reference herein in their entireties, and applicants claim the benefits of the Non provisional applications and the PCT application under 35 U.S.C. §120 and the benefits as to the Australian national application and the U.S. Provisional application under 35 U.S.C. §119.

### FIELD OF THE INVENTION

The invention relates to a method for the evaluation of fertility and monitoring of early pregnancy, fetal development, placental development and function, parturition, and conditions such as pre-eclampsia, intrauterine growth restriction (IUGR), early abortion, abnormal uterine bleeding, endometriosis, and cancers, diseases of the heart, testis or ovary, muscle function, including cardiac muscle, skeletal muscle, lung and the diaphragm. The invention also relates to a method to screen candidate drugs for fertility control or for treatment of the above disorders.

### BACKGROUND

Embryo implantation, the process by which the blastocyst attaches and implants in the uterus, leads to the establishment of an intimate relationship between the embryo and the endometrium. Implantation is one of the most important limiting factors in establishing a successful pregnancy. It is a complex process involving active interactions between the blastocyst and the uterus. The uterus must undergo dramatic morphological and physiological changes to transform itself from a non-receptive to a receptive state. This differentiation process is largely mediated by the coordinated effects of the ovarian hormones, which act through their intracellular receptors to regulate gene expression, and hence to influence cellular proliferation and differentiation. It is also regulated by the blastocyst.

Following implantation, successful placental development is essential for establishing pregnancy. One critical event of normal placental development (placentation) is the widening (remodeling) of the spiral arteries of the mother's uterus that deliver blood into the placenta. This process depends on a specialized placental cell type, extravillous cytotrophoblast (EVT), which is invasive in nature. EVTs migrate into the spiral arteries, replacing the endothelial lining and removing the perivascular smooth muscle, thereby converting the narrow and high-resistance spiral arteries into widened and low-resistance channels. This allows expanded capacity of the uteroplacental circulation to support the growing fetus.

During the initial stages of placentation (before 10-12 weeks of gestation), when the EVTs migrate into the spiral arteries, they form plugs and block the arteries, preventing maternal blood flow to the placenta and creating the hypoxic environment required for fetal organogenesis and the development of other placental cell types. Around 13-14 weeks of gestation, the spiral arteries start to be de-plugged and remodelled, resulting in a dramatic increase in blood flow and oxygen concentration. This oxygen switch is a critical milestone in placentation, signifying that the de-plugging/vessel remodeling has occurred and maternal blood flow to the placenta initiated.

While the details of the exact molecular events occurring in the uterus during these processes are still unknown, in principle it can be predicted that a unique set of genes is up- or down-regulated in a temporally and spatially specific manner. However, given the complexity and the as-yet imprecisely defined molecular mechanism of the processes, many molecules critical for establishing pregnancy are still unidentified.

It is an aim of the present invention to identify genes involved in embryo implantation and/or placentation and to determine genes necessary for a successful pregnancy and hence provide a diagnostic and potential treatment target for fertility disorders involving unsatisfactory embryo implantation or placentation.

### SUMMARY

The inventors used the mouse as a model in a search for hitherto unrecognised molecules which are important in the early stage of implantation. In the mouse on day 4.5 of pregnancy (vaginal plug=day 0), the uterus undergoes dramatic morphological changes in association with cell proliferation and differentiation, leading to the acquisition of a receptive state. This uterine remodelling is associated with an increase in vascular permeability at implantation sites. The inventors hypothesized that the proliferation and differentiation of endometrial cells at this time is associated with up- or down-regulation of a number of genes, many of which are still unknown. To identify uterine genes which are potentially critical for uterine receptivity, they used the technique of RNA differential display (DDPCR) and compared the mRNA expression patterns of implantation and interimplantation sites on day 4.5 of pregnancy.

One of the mRNA molecules identified as being differently regulated between the two sites was found to encode a protein molecule, with a predicted serine protease motif. They isolated the mouse cDNA (SEQ ID NO: 26) encoding this protein, and examined its uterine expression during early pregnancy in the mouse; the protein is up-regulated in the pregnant mouse uterus from day 4.5 and further increased in the implantation site (including the maternal deciduum and the fetus and the placenta) from day 8.5 onwards. The observed expression pattern indicated a role for this protein in implantation, placentation and early pregnancy.

They have also identified and isolated the cDNA encoding the corresponding human enzyme (SEQ ID NO: 31), and found that this encodes a protein with a predicted serine protease motif (here called PRSP, and also denoted as HtrA3), which is expressed in endometrium, decidua and placenta, and also in ovary, heart, and certain other tissues.

Subsequent homology searching using the mouse cDNA sequence (SEQ ID NO: 26) located a protein having some sequence homology with a cDNA sequence deposited by direct submission in GenBank under accession number AY037300 (Matsuguchi, T. and Yoshikai, Y, TASP, a novel mammalian serine protease). The function and expression pattern of the gene were not disclosed or discussed in the GenBank disclosure.

Subsequent homology searching using the amino acid sequence (SEQ ID NO: 33) encoded by the human cDNA sequence located a substantially homologous protein (SEQ ID NO: 3 in WO 00/39149 to Millenium Pharmaceuticals, Inc.). SEQ ID NO: 33 also has significant homology to HtrA type proteins. These proteins were not previously suggested to be involved in embryo implantation, pregnancy, or indeed in any reproductive processes.

Further work described herein was performed to identify the role of this protein in pregnancy and to identify potential uses. Based on the results of this work PRSP is believed to be useful in promoting the implantation of the fertilized egg, development of the placenta and the embryo, and maintenance of pregnancy. Accordingly, PRSP may be utilized in methods of monitoring pregnancy and maintenance of proper implantation, placentation, and intra uterine growth, particularly during early pregnancy, particularly the first trimester of pregnancy. PRSP may be utilized in methods for the diagnosis and/or treatment of a variety of fertility-related conditions or other conditions, including infertility due to luteal phase defect, infertility due to failure of implantation, pre-eclampsia, IUGR, early abortion, abnormal uterine bleeding, endometriosis, cancers and parturition. It may also play a role in muscle function, including those of the heart, skeletal muscle, lung and the diaphragm.

Furthermore, the inventors have demonstrated here that maternal serum levels of HtrA3(PRSP) at the end of the 1^{st} trimester predict subsequent development of preeclampsia, a serious disorder of pregnancy. The inventors have also provided strong experimental evidence for likely molecular explanations underpinning this finding. HtrA3 is maximally produced in the placenta in the 1st trimester and then dramatically down-regulated, especially in syncytiotrophoblast. This ontogeny is reflected in its levels in the maternal circulation. Importantly, HtrA3 is regulated in syncytiotrophoblast by hypoxia, being enhanced by low oxygen and dramatically reduced on re-oxygenation. This correlates with its substantially reduced levels in the placenta and in the maternal blood at the time of the low-to-high “oxygen switch’ in vivo. Thus our finding of elevated maternal serum HtrA3 levels at around 13-14 weeks of pregnancy is consistent with the concept that preeclampsia results from abnormal vessel remodelling and prolonged placental exposure to hypoxia, the root cause of preeclampsia.

In a first aspect, the invention provides a method of detecting, diagnosing, or monitoring conditions which involve a change in PRSP expression, such as infertility caused by inability to achieve or sustain embryo implantation or to sustain pregnancy, or insufficiency of placentation (such as may occur in pre-eclampsia or IUGR), comprising the step of measuring the amount or activity of PRSP in a biological sample from a mammal suffering from or at risk of such a condition. Such conditions involving a change in PRSP expression include but are not limited to pre-eclampsia, intrauterine growth restriction (IUGR), early abortion, abnormal uterine bleeding, endometriosis, cancers, and diseases of the heart, testis or ovaries.

In a further such aspect, the invention provides a method for detecting and monitoring PRSP expression and/or activity, particularly in a pregnant mammal, including a pregnant human, particularly during early pregnancy. Significant comparative changes or differences in PRSP expression or activity in a pregnant female mammal versus a control female mammal indicate a pregnant mammal at risk of a condition including and not limited to miscarriage, pre-eclampsia, and intrauterine growth restriction (IUGR). Significant comparative changes or differences in PRSP expression or activity in a pregnant female mammal versus a control female mammal indicate a pregnant mammal at risk of a not normal pregnancy. In a such aspect, the invention provides a method for detecting and monitoring PRSP expression and/or activity, particularly in a pregnant mammal, including a pregnant human, particularly during early pregnancy, particularly during the first trimester and into the second trimester. In a such aspect, the invention provides a method for detecting and monitoring PRSP expression in a pregnant mammal, particularly a pregnant human, during early pregnancy, particularly 8-20 weeks of pregnancy. In a such aspect, the invention provides a method for detecting and monitoring PRSP expression in a pregnant mammal, particularly a pregnant human, during early pregnancy, particularly 7-15 weeks of pregnancy. In an aspect, the invention provides a method for detecting and monitoring PRSP expression in a pregnant mammal, particularly a pregnant human, during early pregnancy, particularly 7-9 weeks of pregnancy. In a such aspect, the invention provides a method for detecting and monitoring PRSP expression in a pregnant mammal, particularly a pregnant human, during early pregnancy, particularly 8-15 weeks of pregnancy.

The invention provides a method for detecting and monitoring PRSP expression in a pregnant mammal, particularly a pregnant human, during early pregnancy, particularly 7-9 weeks of pregnancy, to determine whether said mammal is a risk of preeclampsia. The invention provides a method for detecting and monitoring PRSP expression in a pregnant mammal, particularly a pregnant human, during early pregnancy, particularly 7-15 weeks of pregnancy, to determine whether said mammal is a risk of IUGR. In an aspect of the method in pregnant humans, human PRSP expression is monitored. In particular, expression of human PRSP SEQ ID NO: 33 or 34 is monitored. Expression of PRSP, including human PRSP may be monitored using an antibody. In particular an antibody raised against PRSP peptide sequence comprising SEQ ID NO: 52 or 56 is used in monitoring PRSP expression.

Any suitable biological sample may be used, for example a tissue or cell sample or extract, or a sample of a biological fluid, such as blood, plasma, serum, amniotic fluid, uterine or bladder washings, urine or saliva.

In a second aspect the invention provides a probe for detection of nucleic acid encoding PRSP, comprising at least 15, preferably at least 20, more preferably at least 30 consecutive nucleotides from a PRSP nucleic acid sequence. In a particularly preferred embodiment the probe encompasses at least part of the common region of the two isoforms disclosed herein for mouse PRSP (SEQ ID NO:40), or human PRSP (nucleotides 1-1243 of the long form sequence shown in SEQ ID NO:31).

Thus the invention in a third aspect provides a method of detecting, diagnosing, or monitoring a condition which involves a change in PRSP expression, comprising the step of using the probe of the second aspect in an assay performed on a biological sample from a mammal suspected to be suffering from such a condition.

Persons skilled in the art would readily appreciate how to assay for PRSP expression using probes against PRSP, including the probes of the invention exemplified herein. Exemplary nucleic acid probes include SEQ ID NO: 40, 50, and 54. Human PRSP expression may be assessed using probes SEQ ID NO: 40, 50 and 54.

In one embodiment of this aspect, total RNA in a sample of placental or uterine tissue from the mammal is assayed for the presence of PRSP messenger RNA, wherein an alteration in the amount of PRSP messenger RNA is indicative of impaired fertility or of impending miscarriage.

It will be appreciated that probes according to the invention may be used to identify genetic polymorphisms which are indicative of predisposition or susceptibility to PSRP-related conditions.

In a fourth aspect the invention provides an antibody directed against PRSP. The antibody may be polyclonal or monoclonal, and is preferably monoclonal. The antibody may suitably be directed against one of the following segments of the mouse protease of SEQ ID NO: 27:
1. Amino acids 133-142; sequence PSGLHQLTSP (SEQ ID NO:51).
2. Amino acids 116-126; sequence ALQVSGTPVRQ (SEQ ID NO:52).
3. A sequence common to both isoforms, represented by amino acids 313-324 of SEQ ID NO:27; sequence GPLVNLDGEVIG (SEQ ID NO:53).

These mouse sequences are highly homologous to corresponding regions of the human protein.

The antibody may suitably be directed against one of the following segments of the human protease of SEQ ID NO: 33:
1. Amino acids 127-136; sequence PLGLHQLSSP (SEQ ID NO:55).
2. Amino acids 110-120; sequence ALQLSGTPVRQ (SEQ ID NO:56).
3. A sequence common to both isoforms, represented by amino acids 307-318 of SEQ ID NO:33; sequence GPLVNLDGEVIG (SEQ ID NO:57).

In one particularly preferred embodiment the antibody has the ability to inhibit the serine protease activity and/or the IGF-binding activity of the PRSP.

Thus the invention in a fifth aspect provides a method of detecting, diagnosing, or monitoring a condition which involves a change in PRSP expression, comprising the step of using the antibody of the fourth aspect in an assay performed on a biological sample from a mammal suspected to be suffering from such a condition.

Persons skilled in the art would readily appreciate how to assay for PRSP protein using the antibody of the invention.

For example, the probes or antibodies of the invention may be used to diagnose impaired fertility or impending miscarriage, as described above. The antibodies of the invention are expected to be particularly useful for detecting PRSP in biological fluids such as blood, plasma, serum or amniotic fluid, in uterine or bladder washings, in saliva, or urine, as described above.

In a sixth aspect the invention provides a method of screening for compounds which have the ability to modulate the activity of PRSP, comprising the step of assessing the ability of a candidate compound to increase or decrease
(a) the serine protease activity and/or
(b) the IGF-binding activity of PRSP.

It will be appreciated that modulation of PRSP activity may be detected inter alia by monitoring the effects of the candidate compound on levels of a substrate for the enzyme, or on a cellular activity of PRSP. The substrate assay may utilise synthetic substrates, and suitable substrates are well known in the art. Assays for cellular activity may utilise cell lines which have been transfected with nucleic acid encoding PSRP so as to over express this protein; such transformed cell lines are particularly useful for phenotypic assays of biological function.

Thus the invention in a seventh aspect provides a method of identifying agonists and antagonists of PRSP. In view of the crucial role of PRSP in implantation and in formation of the placenta indicated by the results reported herein, it is contemplated that antagonists and/or agonists of PRSP will be useful as agents for modulating fertility or for supporting at least the early phases of pregnancy. It is further contemplated that antagonists of PRSP include, but are not limited to, antibodies and anti-sense nucleic acids.

In an eighth aspect the invention provides a null mouse model in which expression of serine protease genes having SEQ ID NO:26, 31, 32, or 38 and therefore serine protease proteins having SEQ ID NO: 27, 33, 34 or 39, is blocked. Preferably, the null mouse has the genes having SEQ ID NO: 26 and or 38 deleted.

While it is particularly contemplated that the compounds of the invention are suitable for use in medical treatment of humans, they are also applicable to veterinary treatment, including treatment of companion animals such as dogs and cats, and domestic animals such as horses, cattle and sheep, zoo animals such as non-human primates, felids, canids, bovids, and ungulates, or for the control of pest or feral species such as rabbits, rats and mice.

### BRIEF DESCRIPTION OF THE FIGURES

FIG. 1A illustrates the results of RNA differential display analysis (DDPCR) of pregnant mouse uterus. The expression pattern of band 10 (identified to be PRSP) on the DDPCR gel is indicated by the arrow, showing much stronger intensities in interimplantation sites (Inter) compared to implantation sites (Imp) in four different mice: lane 1, animal 1; lane 2, animal 2, lane 3, animal 3 and lane 4, animal 4.

FIG. 1B shows the results of Northern blot analysis of mRNA detected using the cDNA extracted from band 10 of the DDPCR gel as a probe. Total RNA (15 μg) was isolated from implantation (Imp) and interimplantation (Inter) sites of day 4.5 pregnant mice. The top panel shows the 2.8 kb band detected for this gene; the lower panel shows the signal detected by the GAPDH probe on the same membrane as in the top panel.

FIG. 2 shows the full length cDNA sequence (SEQ ID NO: 26) and predicted amino acid sequence (SEQ ID NO: 27) of the longer isoform of the novel protein from mouse uterus. The ATG start codon and TGA stop codon are boxed. The 16 cysteine residues are shown in bold and boxed, and the serine protease active site residues GNSGGPL (residues 309-315 of SEQ ID NO: 27) and the additional histidine site residues TNAHV (residues 194-198 of SEQ ID NO: 27) are shown underlined and in bold.

FIG. 3A shows the cDNA sequence of the long isoform encoding the human protease (SEQ ID NO:31; 2543 bp); the start and stop codons are indicated by the box.

FIG. 3B shows the cDNA sequence of the short isoform encoding the human protease (SEQ ID NO:32; 1953 bp); the start and stop codons are indicated by the box.

FIG. 4A shows the deduced amino acid sequence of the long isoform of the human protease (SEQ ID NO:33; 453 amino acids).

FIG. 4B shows the deduced amino acid sequence of the short isoform of the human protease (SEQ ID NO:34; 357 amino acids).

FIG. 5A and FIG. 5B respectively show the comparison between the cDNA and protein sequences of the two isoforms of the human enzyme. In FIG. 5A, the top sequence shows nucleotides 86-1245 of SEQ ID NO: 31 and the bottom sequence shows nucleotides 1-1160 of SEQ ID NO: 32. In FIG. 5B, the top sequence shows residues 1-371 of SEQ ID NO: 33, and the bottom sequence is SEQ ID NO: 34.

FIG. 6A shows the full length cDNA sequence (SEQ ID NO:38) encoding the short isoform of the novel protein from mouse uterus. The ATG start codon and TGA stop codon are indicated by boxes.

FIG. 6B shows the deduced amino acid sequence of the short isoform of the mouse protease (SEQ ID NO:39; 363 amino acids).

FIG. 6C shows a comparison between the deduced amino acid sequences of the longer (top) (residues 1-363 of SEQ ID NO: 27) and shorter (bottom) (SEQ ID NO: 39) isoforms of the mouse enzyme. The 16 cysteine residues are shown in bold and boxed, and the serine protease active site residues GNSGGPL (residues 309-315 of SEQ ID NO: 27) and the additional histidine site residues TNAHV (residues 194-198 of SEQ ID NO: 27) are shown underlined and in bold.

FIG. 7 shows the results of Northern blot analysis of the novel gene in the mouse uterus during early pregnancy. A 785 by cDNA sequence (nt 76-860 of the longer cDNA shown in FIG. 2), representing the common region of the two isoforms, was used as a probe. Total RNA (15 μg) was isolated from whole uterus of non-pregnant mice at estrus (NP) and from whole uterus of 3.5 day pregnant (d3.5) mice, and from implantation sites (Imp) and interimplantation sites (Inter) of uterus on days (d) 4.5, 5.5, 6.5, 8.5 and 10.5 of pregnancy (day 0=day of vaginal plug). On days 8.5 and 10.5, three types of tissue were sampled: (1) the entire implantation unit containing the uterine implantation site, the deciduum, embryo and the developing placenta [Imp (+)], (2) uterine implantation site tissue without the deciduum, embryo and placenta [Imp (−)], and (3) embryo and placenta sampled together (Emb+Pl) on day 8.5, and placenta (Pla) only on day 10.5. The top panel shows the main 2.8 kb transcript detected for this gene, and the lower panel shows the signal detected by the GAPDH probe on the same membrane.

FIG. 8 shows the results of Northern blot analysis of the tissue specificity of the novel gene from mouse. Total RNA (15 μg) was isolated from interimplantation (Inter) and implantation (Imp) sites on day 4.5 pregnancy, placenta on day 10.5, intestine, lung, liver, testis, ovary, heart, spleen, kidney, whole brain and muscle. A 785 by cDNA sequence (nt 76-860 of the longer cDNA shown in FIG. 2) representing the common region of the isoforms was used as a probe. The top panel shows the signals detected for this gene, and the lower panel shows the signal detected by ribosomal 18s RNA probe on the same membrane.

FIG. 9 shows the results of probing a human multi-tissue expression array with the same 785 bp PRSP cDNA probe as in FIG. 7.

FIG. 10 shows the results of Southern blot analysis of the novel gene in the mouse. Genomic DNA was isolated from non-pregnant mouse uterus, and 10 μg was digested with the following four restriction enzymes: TaqI, HindIII, EcoRI and BamHI, and probed with a radio-labelled 785 by cDNA sequence (nt 76-860 of the longer cDNA shown in FIG. 2), representing the common region of the two isoforms.

FIG. 11 shows the results of semiquantitative reverse transcriptase polymerase chain reaction (RT-PCR) Southern blot analysis of HtrA (a related peptide) and PRSP (short and long forms) in cycling and pregnant human endometrium. Menstrual phase endometrium (lanes 1-3), early proliferative phase endometrium (lanes 4-7), mid-late proliferative phase endometrium (lanes 8-9), early secretory phase endometrium (lanes 10-13), mid-late secretory phase endometrium (lanes 14-18), premenstrual endometrium (lanes 19-22), first trimester decidua (lanes 23, 25, 27, 29, 31), first trimester placenta (lanes 24, 26, 28, 30, 32), term placenta (lane 34), pre-menopausal ovary (lane 35), post-menopausal ovary (lane 37), heart (lane 33), and skeletal muscle (lane 36).

FIG. 12 shows the result of in situ hybridization to detect PRSP mRNA in cycling human endometrium on day 9 of the menstrual cycle.

FIG. 13 shows the scheme of antibody generation in the sheep against peptides of mouse PRSP protein. The same scheme could be used to generate antibodies against peptides of human PRSP protein or a similar scheme could be used in another species such as rabbit.

FIG. 14 shows the detection of the antibody in the serum of immunized sheep and in IgG prepared from the serum by dot blot of peptides. The result for peptide (2), identified in Example 10, is shown. To show the specificity of the antisera, dots 1 to 4 contain serial dilutions of peptide (2) and dots 5 and 6 contain irrelevant peptides.

FIG. 15 shows the result of western blot analysis of PRSP protein in the non-pregnant mouse uterus (M np-uterus), mouse placenta on day 10.5 of pregnancy (M-placenta) and human endometrium on day 25 of the menstrual cycle (H-endo), using the antibody against peptide (2).

FIG. 16 shows the result of western blot analysis of PRSP protein in the serum of two pregnant women using the antibody against peptide (2).

FIG. 17 shows the result of Northern analysis of PRSP in a range of human tissues. PBL: peripheral blood leukocytes; S intestine: small intestine; Skel muscle: skeletal muscle.

FIG. 18 shows the result of Northern analysis of PRSP in first trimester pregnant human decidua (D) and placenta (P).

FIG. 19 shows a proposed molecular mechanism for the generation of long and short isoforms of PRSP protein due to alternative splicing of the pre-mRNA in the mouse and human.

FIGS. 20-21 are results from studies in mice in which the HtrA3 gene has been deleted, where +/+ represents wild-type, +/− represents heterozygotes and −/− represents homozygotes.

FIG. 20 shows the effects of maternal phenotype on fetal weight at day 18 of pregnancy.

FIG. 21 shows the effects of maternal phenotype on placenta weight at day 18 of pregnancy.

FIGS. 22-24 are data showing serum levels of PRSP in pregnant women who subsequently did not (normal) or did develop either IUGR (FIG. 22) or preeclampsia (PE) (FIGS. 23, 24) later in gestation

FIG. 22 shows serum levels of the protease (the 39 kD band) at 7-9 weeks of gestation.

FIG. 23 shows serum levels of the protease (the 39 kD band) at 9 weeks of gestation.

FIG. 24 shows serum levels of the protease (the 39 kD band) at 13-14 weeks of gestation.

FIG. 25 shows cellular localization and expression levels of the protease in placental/maternal cells across gestation as determined by immunohistochemical analysis.

FIG. 26 shows serum levels of the protease in maternal blood across gestation in women with normal pregnancies.

FIG. 27 shows protease levels in the media of explant culture of first trimester placenta following exposure to normoxic (20% oxygen) or hypoxic (2% oxygen) conditions.

FIG. 28 shows PRSP protein levels in syncytial BeWo cells when grown in normoxic (20%) or hypoxic (2.5%) environment, or switched from the hypoxic to normoxic (2.5%→20%) conditions.

### DETAILED DESCRIPTION OF THE INVENTION

### PRSP.

Several sequences for serine proteases upregulated at the site of embryo implantation during early pregnancy have been identified and these have substantial sequence homology to proteins of the HtrA family. Suitable PRSP proteins and nucleic acid molecules encoding them are provided as SEQ ID Nos: 26, 27, 31, 32, 33, 34, 38 and 39.

PRSP is also denoted as HtrA3, as a new family member of the HtrA family, and PRSP and HtrA3 may be used herein interchangeably and by reference.

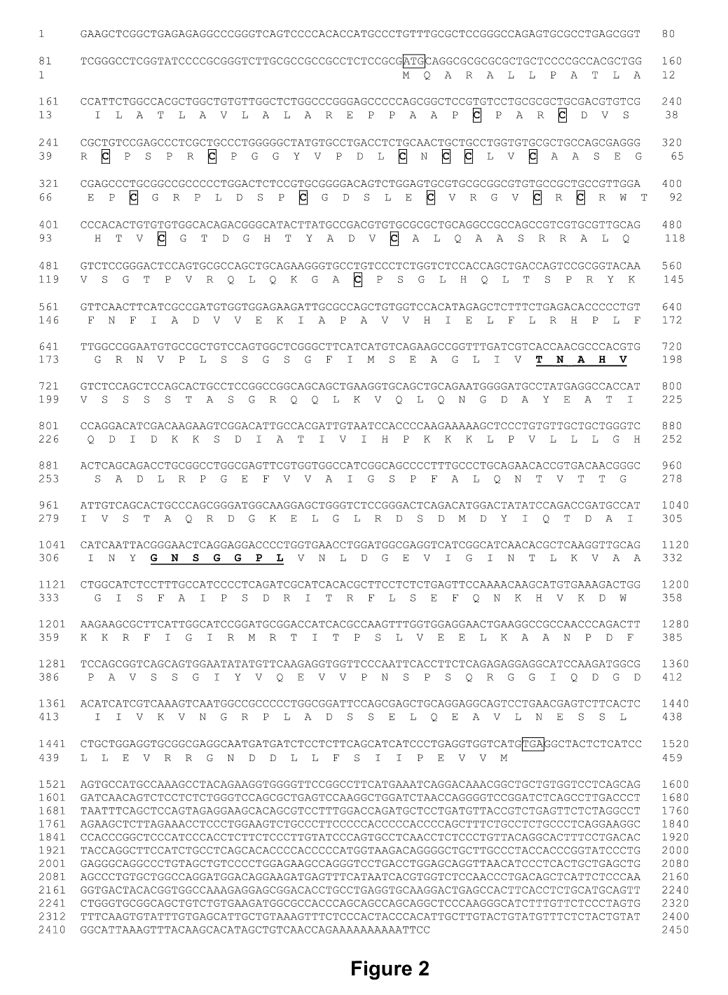

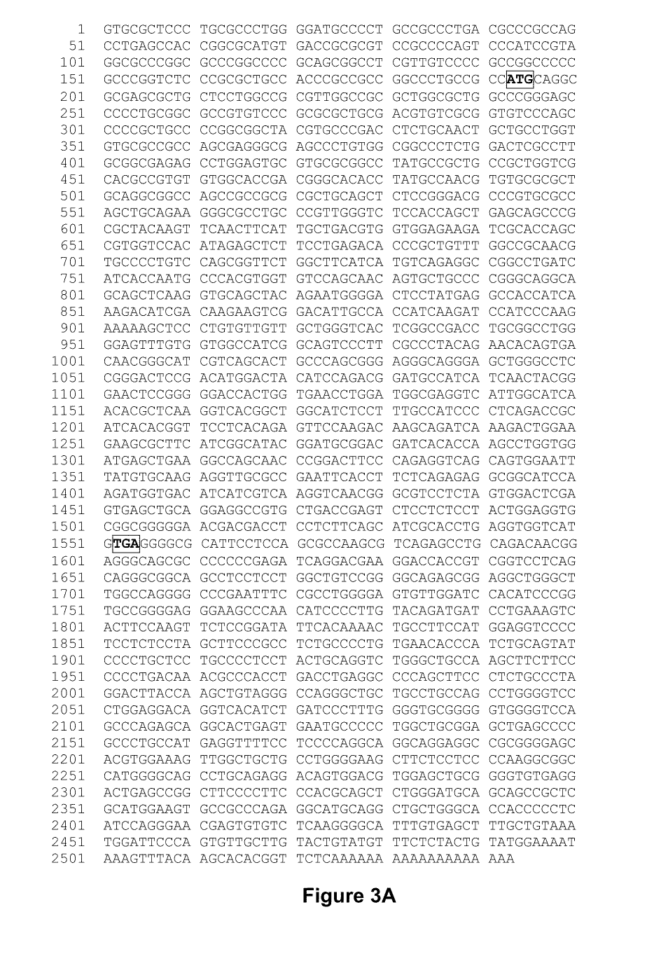

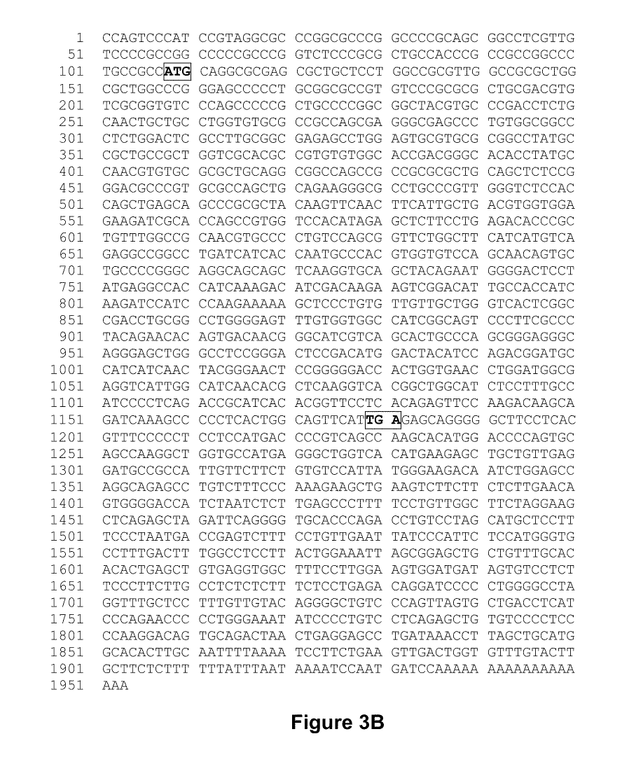

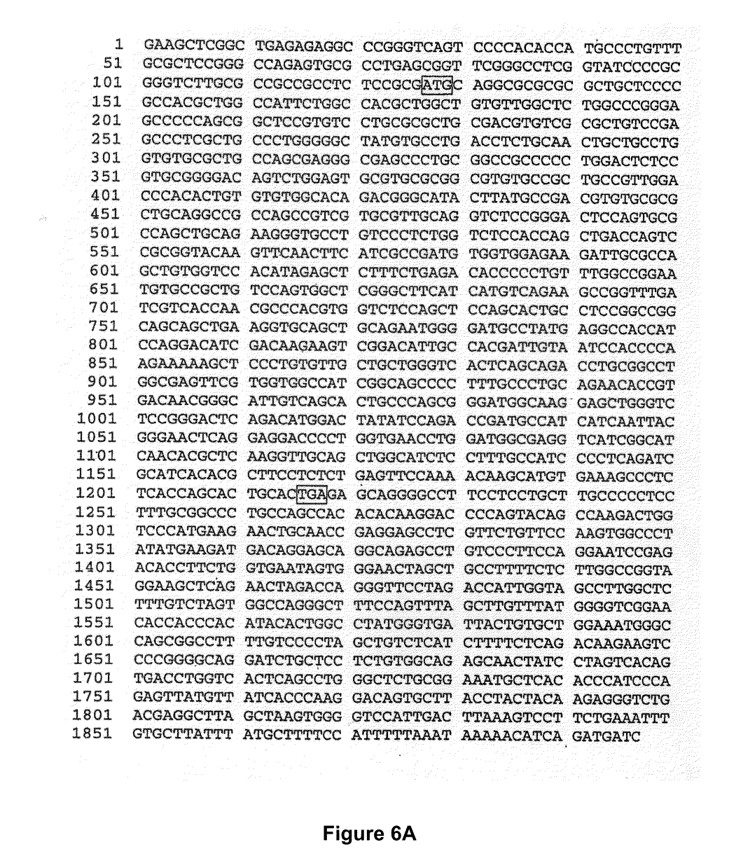

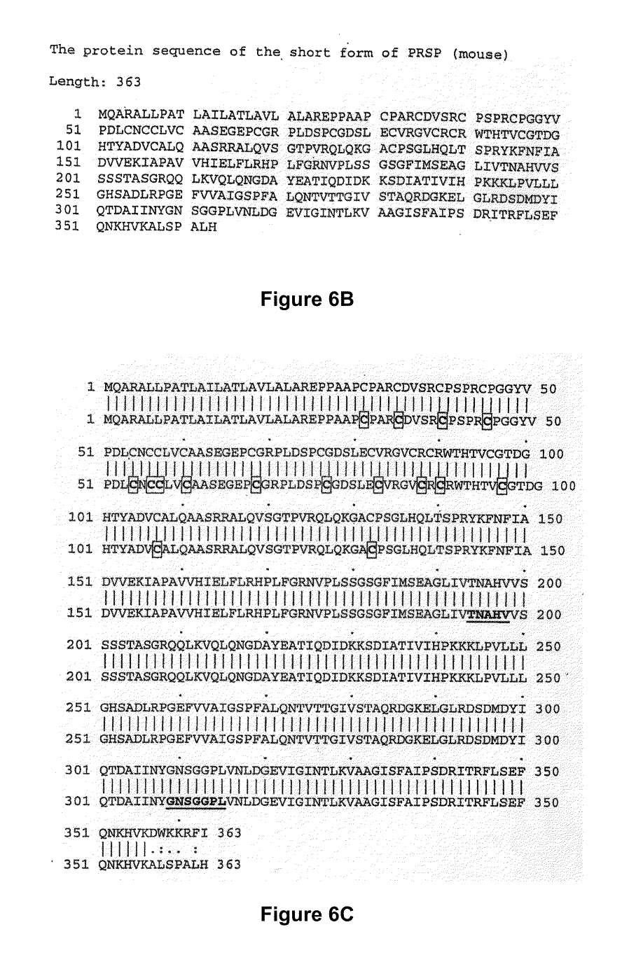

The PRSP nucleic acid molecule may have a sequence selected from the group consisting of
(a) a cDNA molecule having the sequence set out in FIG. 2 (SEQ ID NO:26), FIG. 3A (SEQ ID NO:31), FIG. 3B (SEQ ID NO:32), or FIG. 6A (SEQ ID NO:38);
(b) a nucleic acid molecule which is able to hybridize under at least moderately stringent conditions to the molecule of (a); and
(c) a nucleic acid molecule which has at least 75% sequence identity to the molecule of (a).

More preferably in (b) the nucleic acid molecule is able to hybridize under stringent conditions to the molecule of (a). More preferably in (c) the nucleic acid molecule has at least 80%, even more preferably at least 90% sequence identity to the molecule of (a).

The PRSP protein has serine protease enzymic activity and an IGF-binding motif, and is encoded by the nucleic acid molecule of the invention. This protein is referred to herein as pregnancy-related serine protease (PRSP). It will be clearly understood that all isoforms of PRSP are within the scope of the invention.

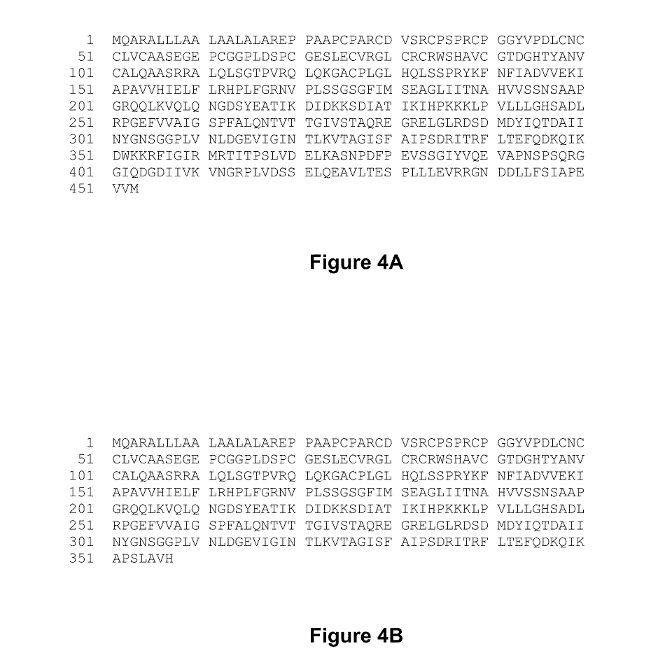

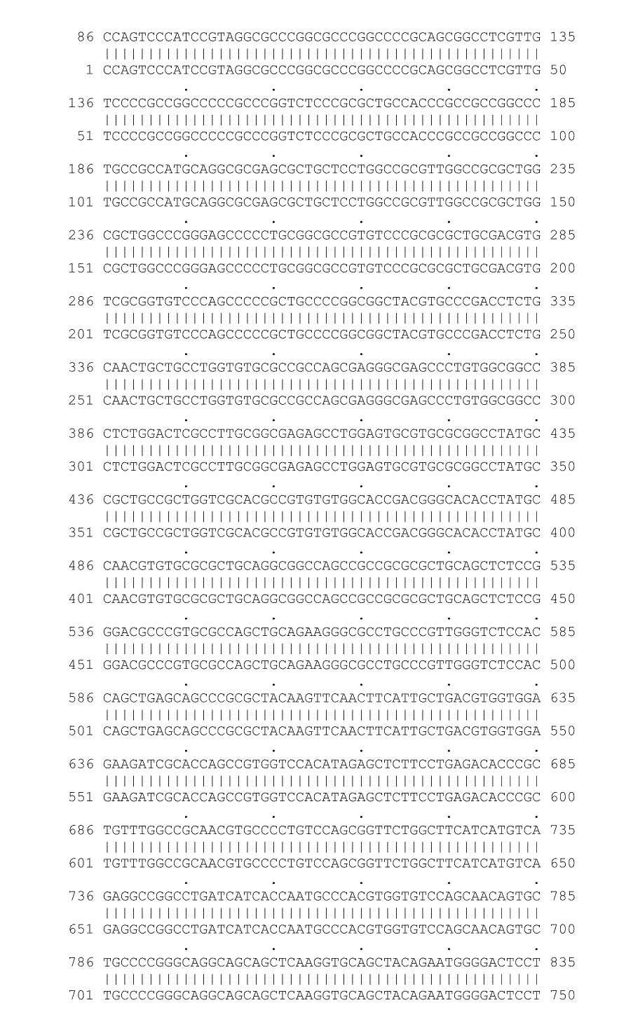

Preferably the protein has a sequence selected from the group consisting of the sequences set out in FIG. 2 (SEQ ID NO:27), FIG. 6B (SEQ ID NO:39), FIG. 4A (SEQ ID NO:33), or FIG. 4B (SEQ ID NO:34); more preferably the sequence is the one set out in FIG. 4A (SEQ ID NO:33) or FIG. 4B (SEQ ID NO:34).

PRSP amino acid sequence variants are included within the definition of PRSP proteins, provided that they are functionally active. As used herein, the terms “functionally active” and “functional activity” in reference to PRSP mean that the PRSP is able to act as a serine protease and/or to bind IGF, and/or that the PRSP is immunologically cross-reactive with an antibody directed against an epitope of a naturally-occurring PRSP of the invention. It will be appreciated that PRSP may also have other biological functions in addition to those specifically mentioned herein.

Therefore PRSP amino acid sequence variants will generally share at least about 75%, preferably greater than 80%, and more preferably greater than 90% sequence identity with one or more of the deduced amino acid sequences set out in in FIG. 2 (SEQ ID NO:27), FIG. 6B (SEQ ID NO:39), FIG. 4A (SEQ ID NO:33), or FIG. 4B (SEQ ID NO:34), after aligning the sequences to provide for maximum homology.

“PRSP nucleic acid” is RNA or DNA which encodes PRSP. “PRSP DNA” is DNA which encodes PRSP. PRSP DNA is obtained from cDNA or genomic DNA libraries, or by in vitro synthesis. Identification of PRSP DNA within a cDNA or a genomic DNA library, or in some other mixture of various DNAs, is conveniently accomplished by the use of an oligonucleotide hybridization probe which is labeled with a detectable moiety, such as a radioisotope. To identify DNA encoding PRSP, the nucleotide sequence of the hybridization probe is preferably selected so that the hybridization probe is capable of hybridizing preferentially to DNA encoding the PRSP amino acid sequence set out in FIG. 2 (SEQ ID NO: 26), FIG. 4A (SEQ ID NO: 33), FIG. 4B (SEQ ID NO: 34) or FIG. 6B (SEQ ID NO: 39), under the hybridization conditions chosen. Preferably the probe sequence is the one encoding the common region of the two isoforms of either the mouse or the human PRSP.

“Isolated” PRSP nucleic acid is PRSP nucleic acid which is identified and separated from, or otherwise substantially free from, contaminant nucleic acid encoding other polypeptides. The isolated PRSP nucleic acid can be incorporated into a plasmid or expression vector, or can be labeled for diagnostic and probe purposes, using a label as described further.

### PRSP Related Disorders

PRSP is believed to be useful in promoting the implantation of the fertilized egg, development of the placenta and the embryo, and maintenance of pregnancy. Accordingly, PRSP may be utilized in methods for the diagnosis and/or treatment of a variety of fertility-related conditions or other conditions, including infertility due to luteal phase defect, infertility due to failure of implantation, pre-eclampsia, IUGR, early abortion, abnormal uterine bleeding, endometriosis, cancers and parturition. It may also play a role in muscle function, including those of the heart, skeletal muscle, lung and the diaphragm.

Infertility or fertility related conditions as described herein include those caused by inability to achieve or sustain embryo implantation or to sustain a normal pregnancy to full term. A normal pregnancy is a pregnancy that runs to full term without the need for medical intervention.

Infertility as used herein includes disorders such as pre-eclampsia and intrauterine growth restriction (IUGR), which may provide healthy offspring, but do involve complications with pregnancy and also includes conditions such as early abortion and abnormal uterine bleeding.

### Assay

In an embodiment of the first aspect the invention provides a method of diagnosing an infertility condition in a human female subject, the method comprising
(a) detecting pregnancy-related serine protease (PRSP) protein in a test sample taken from said subject at between 8 and 20 weeks into pregnancy;
(b) detecting PRSP protein in a control sample from a fertile control human female taken at the same number of weeks into pregnancy in the control as the sample taken from the subject; and
(c) comparing the PRSP protein in the test sample with the PRSP protein detected in the control sample,
   in which a change in the PRSP protein in the test sample compared to the control sample is indicative of an infertility condition.

In an embodiment of the first aspect the invention provides a method of determining whether a pregnant female is at risk of an infertility condition in a human female subject, the method comprising
(a) detecting pregnancy-related serine protease (PRSP) protein in a test sample taken from said subject at between 8 and 20 weeks into pregnancy;
(b) detecting PRSP protein in a control sample from a fertile control human female taken at the same number of weeks into pregnancy in the control as the sample taken from the subject, or using predetermined control levels of PRSP detected in one or more control sample from one or more fertile control human female; and
(c) comparing the PRSP protein in the test sample with the PRSP protein detected in the control sample,
   in which a change or significant difference in the PRSP protein in the test sample compared to the control sample is indicative of the risk of an infertility condition.

In an embodiment of the first aspect the invention provides a method of determining whether a pregnant female is at risk of an infertility condition in a human female subject, the method comprising
(a) detecting pregnancy-related serine protease (PRSP) protein in a test sample taken from said subject in the first and second trimester of pregnancy;
(b) detecting PRSP protein in a control sample from a fertile control human female taken at the same number of weeks into pregnancy in the control as the sample taken from the subject, or using predetermined control levels of PRSP detected in one or more control sample from one or more fertile control human female; and
(c) comparing the PRSP protein in the test sample with the PRSP protein detected or predetermined in the control sample,
   in which a change or significant difference in the PRSP protein in the test sample compared to the control sample is indicative of the risk of an infertility condition.

Samples upon which to assay for PRSP may be taken from a pregnant mammal early in pregnancy, particularly the first trimester. The sample may be taken at least once, and possibly more than once, during 7-20 or 8-20 weeks of pregnancy, including 7-15 weeks, 8-15 weeks, 8-14 weeks, 7-9 weeks, 8-9 weeks, 8-10 weeks, 13-14 weeks and 9 weeks. In an aspect, the period(s) between 8-14, 8-10 and 8-9, or 13-14 weeks being particularly preferred. From experimental evidence, the best time to take a sample upon which to assay for PRSP is 8-20 weeks, with the period between 8-14, 8-10 and 8-9, or 13-14 weeks being particularly preferred. Tests performed on samples taken at 9 weeks into pregnancy have been shown to be able to diagnose between IUGR and pre-eclampsia. Control samples may be taken from nonpregnant or pregnant women at comparable times in the menstrual cycle. Experimental samples may be compared with controls taken and assayed simultaneously, assayed simultaneously, or assayed historically and the historical data used for comparison. Further, it should be recognized that the number of weeks of pregnancy are estimated including based on the pregnant female's cycle, her information as provided, testing of hormone levels for estimation, ultrasound data, etc and may not be precise.

In an embodiment of the invention methods, PRSP protein in the test and/or control samples is indicated by a PRSP band on Western blot using an antibody raised against SEQ ID NO: 52 or SEQ ID NO: 56. In one such embodiment, human or mouse PRSP protein in the test and/or control samples is indicated by a PRSP band on Western blot using an antibody raised against SEQ ID NO: 52 or SEQ ID NO: 56. Human PRSP protein in the test and/or control samples is indicated by a positive band on Western blot or antibody bound protein in other antibody assay methods using an antibody raised against SEQ ID NO: 52 or SEQ ID NO: 56.

In one aspect thereof, PRSP protein in the test and, or control samples is indicated by a PRSP band on Western blot or antibody bound protein in other antibody assay methods using an antibody raised against SEQ ID NO: 52 or SEQ ID NO: 56. A relative difference, decrease or increase, in PRSP in a pregnant female versus a control indicate risk of a not normal pregnancy or a pregnancy condition. A decrease in PRSP in a pregnant female, or significantly less PRSP versus a control, as assessed by antibody binding indicates a risk of IUGR. In particular, a decrease in PRSP at weeks 8-15 of pregnancy in a pregnant female indicates a risk of IUGR. An increase in PRSP in a pregnant female, or significantly more PRSP versus a control, as assessed by antibody binding indicates a risk of preeclampsia (PE). In particular, an increase in PRSP at week 7-9, particularly week 9, of pregnancy in a pregnant female indicates a risk of PE. In an aspect thereof human PRSP is measured using antibody, particularly human PRSP of SEQ ID NO: 33 and/or SEQ ID NO:34.

In an embodiment of the first aspect, PRSP protein in the test and, or control samples is indicated by a 39 kDa PRSP band on Western blot using an antibody raised against SEQ ID NO: 52 or SEQ ID NO: 56.

In one embodiment of the first aspect, the change in PRSP protein is indicated by a decrease in the density of the 39 kDa PRSP band, indicative of IUGR.

In another embodiment of the first aspect, the change in PRSP protein is indicated by an increase in the density of the 39 kDa PRSP band, indicative of pre-eclampsia.

In another embodiment of the first aspect, the PRSP protein is detected using an antibody. In one embodiment the antibody is raised against a sequence specific for PRSP, such as SEQ ID NO:51 or 52, or amino acids 133 to 142 or 116 to 126 of SEQ ID NO:33, SEQ ID NO: 55 or 56.

Peptides specific for PRSP may be utilized as a target in the assay. These may have a minimum of 6 contiguous amino acids specific for a PRSP sequence, preferably a PRSP sequence selected from SEQ ID NO: 27, 33, 34 or 39. Peptides having 10, 20, 30, 50 or 100 residues are particularly targeted.

For diagnostic applications, anti-PRSP antibodies typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; radioactive isotopic labels, such as, e.g., ¹²⁵I, ³²P, ¹⁴C, or ³H, or an enzyme, such as alkaline phosphatase, beta-galactosidase or horseradish peroxidase.

Any method known in the art for separately conjugating the antibody to the detectable moiety may be employed. The anti-PRSP antibodies may be employed in any known assay method, such as competitive binding assays, direct and indirect sandwich assays, and immunoprecipitation assays.

PRSP may be used as an immunogen to generate anti-PRSP antibodies. Preferably the PRSP which is used for immunization comprises the region of the PRSP molecule which is common to the two isoforms described herein. Such antibodies, which specifically bind to PRSP, are useful in assays for PRSP, such as in a radioimmunoassay, enzyme-linked immunoassay, or competitive-type receptor binding assays, radioreceptor assay, as well as in affinity purification techniques.

Polyclonal antibodies directed toward PRSP are generally raised in animals by multiple subcutaneous or intraperitoneal injections of PRSP and an adjuvant. If necessary, immunogenicity may be increased by conjugating PRSP or a peptide fragment thereof to a carrier protein which is immunogenic in the species to be immunized.

Monoclonal antibodies directed toward PRSP are produced using any method which provides for the production of antibody molecules by continuous cell lines in culture. The modifier “monoclonal” indicates the character of the antibody as being obtained from a substantially homogeneous population of antibodies, and is not to be construed as requiring production of the antibody by any particular method. Examples of suitable methods for preparing monoclonal antibodies include the original hybridoma method of Kohler et al., (1975), and the human B-cell hybridoma method (Kozbor, 1984; Brodeur et al., 1987).

In a preferred embodiment, the anti-PRSP antibody is a “humanized” antibody. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain.

Gene amplification and/or gene expression may be measured in a sample directly, for example, by conventional Southern blotting to quantitate DNA, or by Northern blotting to quantitate mRNA, using an appropriately labeled oligonucleotide hybridization probe, based on the sequences provided herein. Various labels may be employed. However, other techniques may also be employed, such as using biotin-modified nucleotides for introduction into a polynucleotide. The biotin then serves as the site for binding to avidin or antibodies, which may be labeled with a wide variety of labels, such as radioisotopes, fluorophores, chromophores, or the like. Alternatively, antibodies which can recognize specific duplexes, including DNA duplexes, RNA duplexes, and DNA-RNA hybrid duplexes or DNA-protein duplexes, may be employed. The antibodies in turn may be labelled, and the assay may include a step in which the duplex is bound to a surface, so that upon the formation of duplex on the surface, the presence of antibody bound to the duplex can be detected.

Gene expression may alternatively be measured by immunological methods, such as immunohistochemical staining of tissue sections and assay of cell culture or body fluids, to quantitate directly the expression of the gene product, PRSP. With immunohistochemical staining techniques, a tissue or cell sample is prepared, typically by dehydration and fixation, followed by reaction with labelled antibodies specific for the gene product coupled, where the labels are usually visually detectable, such as enzymatic labels, fluorescent labels, luminescent labels, and the like. Antibodies useful for immunohistochemical staining and/or assay of sample fluids may be either monoclonal or polyclonal. Conveniently, the antibodies may be prepared against a synthetic peptide based on the DNA sequences provided herein.

Oligonucleotides for use as probes or primers may be prepared by any suitable method, such as by purification of a naturally-occurring DNA or by in vitro synthesis. The general approach to selecting a suitable hybridization probe or primer is well known. Typically, the hybridization probe or primer will contain 10-25 or more nucleotides, and will include at least 5 nucleotides on either side of the sequence encoding the desired mutation so as to ensure that the oligonucleotide will hybridize preferentially to the single-stranded DNA template molecule.

In another embodiment of the first aspect, the biological sample or test sample is a sample of a biological fluid, such as plasma, serum, uterine or bladder washings, or amniotic fluid, blood or urine, or a tissue or cellular sample or extract thereof, such as placental or uterine tissue.

### PRSP Agonists and Antagonists and Treatment of PRSP Related Disorders

“PRSP antagonist” or “antagonist” refers to a substance which opposes or interferes with a functional activity of PRSP. PRSP antagonists include, but not limited to, PRSP antibodies, including neutralizing antibodies, and antisense to PRSP.

“PRSP agonist” refers to a substance that induces or enhances the functional activity of PRSP. PRSP is a PRSP agonist.

Isolated PRSP nucleic acid may be used to produce PRSP by recombinant DNA and recombinant cell culture methods for production of PRSP in large quantities.

Neutralizing anti-PRSP antibodies are useful as antagonists of PRSP. The term “neutralizing anti-PRSP antibody” as used herein refers to an antibody which is capable of specifically binding to PRSP, and which is capable of substantially inhibiting or eliminating the functional activity of PRSP in vivo or in vitro. Typically a neutralizing antibody will inhibit the functional activity of PRSP by at least about 50%, and preferably greater than 80%, as determined, for example, by an enzyme activity assay. Neutralising antibodies may act as antagonists of PRSP and thus be useful in treating disorders where a reduction in PRSP is desired.

PRSP agonists and antagonists may be formulated with other ingredients such as carriers and/or adjuvants, e.g. albumin, nonionic surfactants and other emulsifiers. There are no limitations on the nature of such other ingredients, except that they must be pharmaceutically acceptable, efficacious for their intended administration, and cannot degrade the activity of the active ingredients of the compositions. Suitable adjuvants include collagen or hyaluronic acid preparations, fibronectin, factor XIII, or other proteins or substances designed to stabilize or otherwise enhance the active therapeutic ingredient(s).

Animals or humans may be treated in accordance with this invention. It is possible but not preferred to treat an animal of one species with PRSP of another species.

PRSP and PRSP antagonists to be used for in vivo administration must be sterile. The PRSP or PRSP antagonist may be lyophilized to produce sterile PRSP or anti-PRSP antibody in a powder form.

Methods for administering PRSP and PRSP antagonists in vivo include injection or infusion by intravenous, intraperitoneal, intracerebral, intrathecal, intramuscular, intraocular, intraarterial, intrauterine, intracervical, intravaginal or intralesional routes, and by means of sustained-release formulations or by topical application to the skin.

An effective amount of PRSP or PRSP antagonist, e.g., anti-PRSP antibody, to be employed therapeutically will depend upon the therapeutic objectives, the route of administration, and the condition of the patient. Accordingly, it will be necessary for the therapist to titrate the dosage and modify the route of administration as required to obtain the optimal therapeutic effect. A typical daily dosage might range from about 1 μg/kg to up to 100 mg/kg or more, depending on the factors mentioned above. Where possible, it is desirable to determine appropriate dosage ranges first in vitro, for example by using assays for serine protease activity and IGF binding activity which are known in the art, and then in suitable animal models, from which dosage ranges for human patients may be extrapolated.

The invention will now be described in detail by way of reference only to the following non-limiting examples and drawings.

### Materials and Methods

### Animals and Tissue Preparation

Swiss outbred mice were housed and handled according to the Monash University animal ethics guidelines on the care and use of laboratory animals. All experimentation was approved by the Institutional Animal Ethics Committee at the Monash Medical Centre. Adult female mice (6-8 weeks old) were mated with fertile males of the same strain to produce normal pregnant animals, or mated with vasectomized males to produce pseudopregnant mice. The morning of finding a vaginal plug was designated as day 0 of pregnancy. Uterine tissues were collected from non-pregnant mice, or from pregnant mice on days 3-11. A selection of other mouse organs was also collected from non-pregnant mice. Tissues were snap-frozen in liquid nitrogen for Northern analysis, or fixed in 4% buffered formalin (pH 7.6) for in situ hybridization.

For non-pregnant and day 3.5 pregnant mice, the entire uterus was collected. For day 4.5 pregnant mice, implantation sites were visualised by intravenous injections of a Chicago Blue dye solution (1% in saline, 0.1 ml/mouse) into the tail vein 5 min before killing the animals; implantation sites were separated from interimplantation sites, and both sites were retained. For pregnant mice on day 5.5 onwards, implantation and interimplantation sites were visualized without dye injection.

For non-pregnant mice, the uterus was also collected from different stages of the estrous cycle: metestrus, diestrus, proestrus and estrus. The stages of the cycle were determined by analysis of vaginal smears. For ovarian hormone treatments, the animals were first ovariectomized under anaesthesia with avertin, without regard to the stage of the estrous cycle. The animals were allowed to rest for two weeks, then treated with daily subcutaneous injections (0.1 ml per mouse) of steroid hormones (Sigma Chemical Co., St Louis, Mo., USA) for 3 days, as follows: 17β-estradiol (100 ng), progesterone (1 mg), or a combination of both hormones. The steroids were initially dissolved in minimal amounts of ethanol before dilution in peanut oil. Animals injected with oil alone served as controls. Mice were killed 24 h after the last injection.

### Northern Analysis

For Northern analysis, no attempt was made to separate the embryos from the decidua before day 8 of pregnancy, but for 8- and 11-day pregnant mice, embryos were separated from the uterine tissue. Total RNA was extracted from whole uteri or from pools of implantation or inter-implantation sites by the acid guanidinium thiocyanate-phenol-chloroform extraction (GTC) method. RNA (10-15 μg) was denatured at 50° C. for 60 min in 50% dimethylsulfoxide (DMSO) and 1M glyoxal, and the denatured RNA was fractionated by electrophoresis through a 1.2% agarose gel in 10 mM sodium phosphate buffer (pH 7.0) and transferred to positively charged nylon membranes (Hybond-N+, Amersham) by overnight capillary blotting in 5×SSPE (1×SSPE=150 mM NaCl, 10 mM NaH₂PO₄, 1 mM EDTA, pH 7.4). Membranes were baked at 80° C. for 2 h followed by 3 min UV cross linking. Transcript size was estimated by comparison with RNA size standards (Gibco-BRL, Gaithersburg, Md. USA). A simplified filter paper sandwich blotting method was used for the hybridization process at 42° C. overnight, without a prehybridization step. The radio-labelled cDNA probes were generated by random primer labelling of 25 ng cDNA with [³²P]deoxy-CTP (50 μCi/reaction). Unincorporated nucleotides were removed with a MicroSpin S-200 HR column (AMRAD Pharmacia Biotech, Melbourne, Australia). Following hybridization, the blots were rinsed twice with 5×SSPE at 37° C., then twice for 15 min each at 37° C. with 2×SSC/0.1% SDS (w/v) (1×SSC=150 mM NaCl, 15 mM Na₃citrate, pH 7.4). In some cases, additional washes were also performed with 0.5 or 1×SSC/0.1% SDS for 15 min at 60° C. To determine lane to lane loading variation, each blot was also probed with a mouse cDNA probe for glyceraldehyde-3-phosphate dehydrogenase (GAPDH) or 18S ribosomal RNA. Between hybridizations, blots were stripped by incubation at 80° C. for 3 h in 1 mM EDTA/0.1% SDS followed by rinsing in H₂O.

### RT-PCR and T/A Cloning

For reverse transcriptase-polymerase chain reaction (RT-PCR), 1 μg DNA-free total RNA was reverse-transcribed at 46° C. for 1-1.5 h in 20 μl reaction mixture, using 100 ng random hexanucleotide primers and AMV reverse transcriptase (Boehringer-Mannheim, Nunawading, Vic., Australia) with the cDNA synthesis buffer. The PCR was performed in a total volume of 40 μl with 1-1.5 μl of the RT reaction, 1×PCR buffer, 20 μM dNTPs, 10 pmol forward and reverse primers and 2.5 units of Taq DNA polymerase (Boehringer-Mannheim), in 3 stages as follows:
(a) one cycle of an incubation for 5 min at 95° C., 1 min at 52° C.-60° C., and 2 min at 72° C.;
(b) 32 cycles with a denaturation for 45 sec at 95° C., annealing at 52° C.-60° C. for 50 sec and extension at 72° C. for 1 min; and
(c) incubation for 5 min at 72° C.

The PCR products were analysed on 1.5% agarose gel and stained with ethidium bromide. Bands of interest were cut out from the agarose gels, purified with the Qiaquick gel extraction kit (Qiagen Pty Ltd., Clifton Hill, Vic., Australia), cloned into a pGEM-T easy vector (Promega) according to the manufacturer's instructions and sequenced on an automated sequencer (Applied Biosystems, ABI Prism™, 377 DNA Sequencer) using the ABI Prism BigDye terminator cycle sequencing ready reaction kit.

### EXAMPLE 1

### DDPCR Analysis and Identification of Clone 10.9 by Northern Blotting

To identify genes which are potentially critical for the initial process of embryo implantation in the mouse, we compared the uterine gene expression pattern of implantation and inter-implantation sites in the mouse uterus on day 4.5 of pregnancy, using the DDPCR technique. A few bands for which the intensities were different between the two sites were detected on DDPCR gels (Nie et al., 2000b). One of these bands, band 10, was fully analysed, and is described herein.

DDPCR was performed as previously described (Nie et al., 2000b) and was essentially as described originally by Liang and Pardee (1992, 1993). DNA-free RNA from the implantation and interimplantation sites was used as the template for the first-strand cDNA synthesis. The cDNA was then amplified by PCR using one random primer (10 mer) and one oligo-d_{T} anchored primer in the presence of ³³P-dATP. The PCR products were subsequently analysed on 6% high-resolution polyacrylamide/urea gel, and visualised by autoradiography.

Uterine mRNA expression on day 4.5 of pregnancy was compared between implantation sites and inter-implantation sites. The 80 PCR primer combinations (20 random 10 mers combined with 4 oligo-dT anchored primers) used in the DDPCR analysis are shown in Table 1.

TABLE 1
The 80 (4 × 20) primer combinations used in the
DDPCR analysis

| | | |
|---|---|---|
| Primer | Code | Sequence |

3′ primers: Oligo-(dT) anchored primers,
custom-made

| | | | |
|---|---|---|---|
| 1 | T12MA | TTTTTTTTTTTT(G,A,C)A | (SEQ ID NO: 1) |
| 2 | T12MC | TTTTTTTTTTTT(G,A,C)C | (SEQ ID NO: 2) |
| 3 | T12MG | TTTTTTTTTTTT(G,A,C)G | (SEQ ID NO: 3) |
| 4 | T12MT | TTTTTTTTTTTT(G,A,C)T | (SEQ ID NO: 4) |

| | | | |
|---|---|---|---|
| Primer | Code | Sequence | SEQ ID |

5′ Primers: 10 mers, from OPERON

| | | | |
|---|---|---|---|
| 1 | OPA-01 | CAGGCCCTTC | No. 5 |
| 2 | OPA-02 | TGCCGAGCTG | No. 6 |
| 3 | OPA-03 | AGTCAGCCAC | No. 7 |
| 4 | OPA-04 | AATCGGGCTG | No. 8 |
| 5 | OPA-05 | AGGGGTCTTG | No. 9 |
| 6 | OPA-06 | GGTCCCTGAC | No. 10 |
| 7 | OPA-07 | GAAACGGGTG | No. 11 |
| 8 | OPA-08 | GTGACGTAGG | No. 12 |
| 9 | OPA-09 | GGGTAACGCC | No. 13 |
| 10 | OPA-10 | GTGATCGCAG | No. 14 |
| 11 | OPA-11 | CAATCGCCGT | No. 15 |
| 12 | OPA-12 | TCGGCGATAG | No. 16 |
| 13 | OPA-13 | CAGCACCCAC | No. 17 |
| 14 | OPA-14 | TCTGTGCTGG | No. 18 |
| 15 | OPA-15 | TTCCGAACCC | No. 19 |
| 16 | OPA-16 | AGCCAGCGAA | No. 20 |
| 17 | OPA-17 | GACCGCTTGT | No. 21 |
| 18 | OPA-18 | AGGTGACCGT | No. 22 |
| 19 | OPA-19 | CAAACGTCGG | No. 23 |
| 20 | OPA-20 | GTTGCGATCC | No. 24 |

To avoid embryonic contamination, the embryos were removed from the implantation sites under light microscope visualization. After the DDPCR analysis, the differential display pattern was further verified by Northern blotting analysis, and cDNAs from the confirmed bands were sub-cloned into the pGEM-T vector (Promega, Madison, Wis., USA) and sequenced manually.

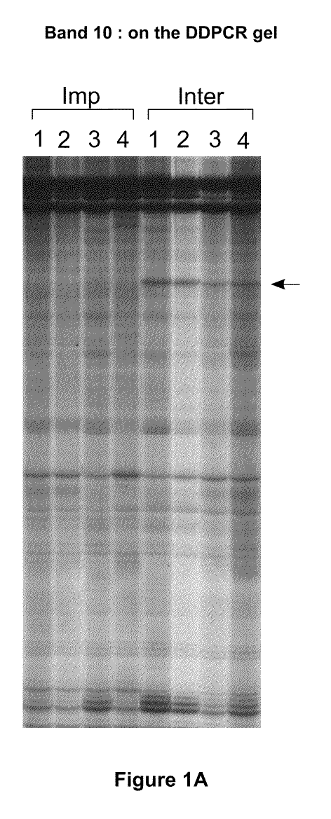

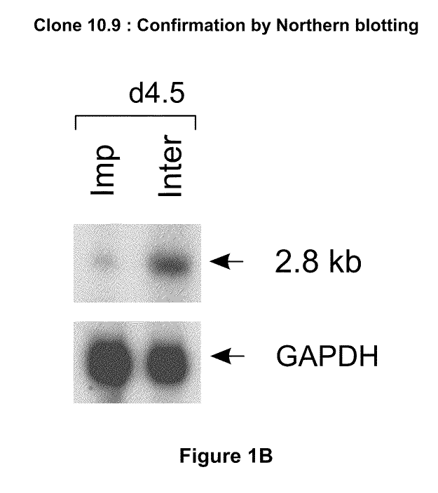

On the DDPCR gel, band 10 was much more intense in interimplantation sites compared to implantation sites in all individual animals tested, as shown in FIG. 1A. To verify that this band indeed represents gene(s) which are differentially expressed between the two sites, the cDNA products of band 10 were extracted from the DDPCR gel, re-amplified, and cloned into the pGEM-T vector, and Northern blot analysis was performed using the cloned inserts as probes. Among the 10 clones analysed, the cDNA of clone 10.9 specifically detected differential expression of mRNA between the two sites on day 4.5 of pregnancy on the Northern blot, with much higher mRNA levels present in interimplantation sites than in implantation sites; this is illustrated in FIG. 1B. A 2.8 kb transcript was detected on this initial blot. This confirmed that clone 10.9 contained the cDNA representing the original expression pattern of band 10 on the DDPCR gel. Of the other clones analysed, clones 10.2 and 11.2 showed results similar to the DDPCR results.

### EXAMPLE 2

### Sequence Analysis of Clone 10.9

Band 10 resulted from the DDPCR amplification of day 4.5 interimplantation site mRNA with the following two primers: 5′ primer, TCTGTGCTGG (OPA-14; SEQ ID NO:18) and 3′ primer, T12MG (SEQ ID NO:3), whose sequences are set out in Table 1 above. After confirming that clone 10.9 contained the cDNA representing band 10, the nucleotide sequence of this clone was determined, and is set out in SEQ ID NO:25.

TABLE 2

The sequence of clone 10.9 (359 bp) derived from band
10 of DDPCR gel (SEQ ID NO: 25)
(The underlined nucleotides represent the primers used
during DDPCR amplification)

| | |
|---|---|
| 1 | TCTGTGCTGG CCAGGATGGA CAGGAAGATG AGTTTCATAA TCACATGGTC |
| 51 | TCCAACCCTG ACAGCTCATT CTCCCAAGGT GACTACACGG TGGCCAAAGA |
| 101 | GGAGCGGACA CCTGCCTGAG GTGCAAGGAC TGAGCCACTT CACCTCTGCA |
| 151 | TGCAGTTCTG GGTGCGGCAG CTGTCTATGA AGATGGCGCC ACCCAGCAGC |
| 201 | CAGCAGGCTC CCAAGGGCAT CTTTGTTCTC CCTAGTGTTT CAAGTGTATT |
| 251 | TGTGAGCATT GCTGTAAAGT TTCTCCCACT ACCCACATTG CTTGTACTGT |
| 301 | ATGTTTCTCT ACTGTATGGC ATTAAAGTTT ACAAGCACAT AGCTGCCAAA |
| 351 | AAAAAAAAA |

This sequence contained 359 nucleotides, and the ends of the sequence indeed contained the unique and expected primer sequences of TCTGTGCTGG at the 5′ end and the reverse complementary sequence of T12MG at the 3′ end (underlined in Table 2). This confirmed that the cDNA in clone 10.9 was the direct PCR product amplified from the specific primers applied during DDPCR amplification.

When compared to the GenBank database, no other sequences were found to be very homologous to clone 10.9, other than a few short expressed sequence tags (ESTs) from mouse uterus, mouse mammary gland, rat mast cell protein 6, rat PC-12 cells, and mouse skin, indicating that this clone represents a novel cDNA sequence.

### EXAMPLE 3

### Cloning of the Full Length cDNA Sequence

In order to obtain the full length cDNA sequence represented by clone 10.9, a mouse uterine cDNA library (Clontech, Palo Alto, Calif.) was screened using radiolabelled clone 10.9 cDNA as a probe; this was prepared as described above for Northern analysis, using standard methods.

Three clones were obtained; all of these appeared to lack the start codon, so 5′ RACE was used in order to obtain the 5′ end sequence. To obtain the full length cDNA sequence and to search for possible isoforms, standard 5′ and 3′ rapid amplification of cDNA ends (RACE) was also performed, using the 5′/3′ RACE kit (Roche, Castle Hill, NSW, Australia).

The longest sequence obtained from these approaches contained 2450 nucleotides, and is shown in FIG. 2 and SEQ ID NO:26. This sequence included an open reading frame of 1377 bp, with the start codon ATG being at nucleotide (nt) 127-129 and the stop codon TGA at nt 1504-1506. It also included a G/C-rich (72%) 5′ untranslated region of 126 by and a 3′ untranslated region of 944 by (FIG. 2).

The open reading frame could be translated into an amino acid sequence of 459 residues (FIG. 2 and SEQ ID NO:27). The predicted protein had a molecular mass of about 49 kDa, with a calculated isoelectric point of 7.08. The N-terminal end of the sequence contained a long stretch of hydrophobic region which may represent a signal peptide.

A comparison of the cDNA and deduced protein sequences with all entries in the GenBank and Swissprot databanks revealed that the most similar entries in the database were human and mouse HtrA. At the cDNA level, this sequence is 63% identical to the mouse (Accession No.: AF172994) and 65% to the human (2 entries, Accession No.: D87258 and Y07921) HtrA cDNA sequences. At the protein level, it is 56% identical to the mouse (Accession No.: AAD49422) and 58% to the human (2 entries, Accession Nos.: BAA13322 and CAA69226) HtrA proteins.

As noted for the human HtrA, this protein also has a substantial similarity to the family of IGF-binding proteins. In particular, the 16 cysteine residues which are conserved in all IGF-binding proteins are present in this protein as well; thus it is expected that the N-terminal of this novel protein represents an IGF-binding domain. The C-terminal part of this protein is closely related to the mouse and human HtrA, which was found to be homologous to the HtrA/Do proteases from bacteria. These HtrA proteins belong to a family of serine proteases which possess the amino acid sequence GNSGGAL (SEQ ID NO:28; in bacterial HtrA) or GNSGGPL (SEQ ID NO:29; in mammalian HtrA) in their active sites, and another TNAHV (SEQ ID NO:30) sequence in the vicinity of the active site. Interestingly, the serine protease active site sequence GNSGGPL was found at position 309-315, and the additional TNAHV residues was located at position 194-198 in this novel protein as shown in FIG. 2. Therefore, we believe that the protein represents a functional serine protease. We conclude that we have isolated cDNA which codes for a serine protease with an IGF-binding motif.

Subsequent homology searching using the mouse cDNA sequence (SEQ ID NO: 26) located a protein having some sequence homology with a cDNA sequence deposited in GenBank under accession number AY037300 (Matsuguchi, T. and Yoshikai, Y, TASP, a novel mammalian serine protease). The function and expression pattern of the gene were not discussed in the disclosure.

### EXAMPLE 4

### Isolation of the Human Protease

Using a 785 by probe (nucleotide 76-860 of the cDNA shown in FIG. 2) derived from the mouse cDNA sequence described in Example 2 as a probe, a human multiple tissue expression array (MTE) (Clontech, Palo Alto, Calif.) was screened, and the heart was identified as one of the most strongly positive tissues. A human heart cDNA library, in which cDNAs were cloned unidirectionally into the Uni-ZAP XR vector (Stratagene Cat # 937257) was screened, using two probes derived from the mouse sequence. Probe 1 contained nucleotide 76-484 and probe 2 contained nucleotide 621-1540 of the mouse cDNA sequence shown in FIG. 2. Three clones were obtained, and all contained the full open reading frames. Of these three clones, two were identical; the three clones were found to represent two different isoforms, and the two cDNA sequences are presented in FIG. 3A (SEQ ID NO:31; long form, 2543 bp) and FIG. 3B (SEQ ID NO:32; short form, 1953 bp) respectively. These two cDNAs code for two proteins: the long isoform codes for a protein of 453 amino acids and the short isoform codes for a protein of 357 amino acids, whose sequences are set out in FIG. 4A (SEQ ID NO:33) and FIG. 4B (SEQ ID NO:34) respectively.

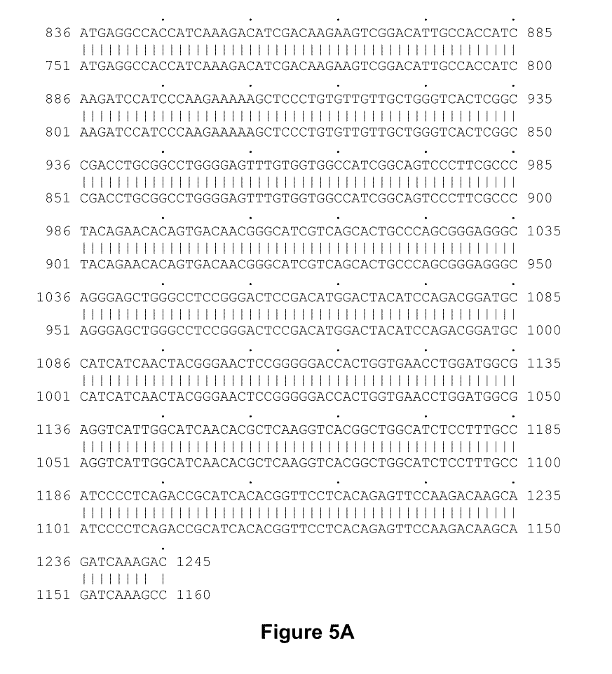

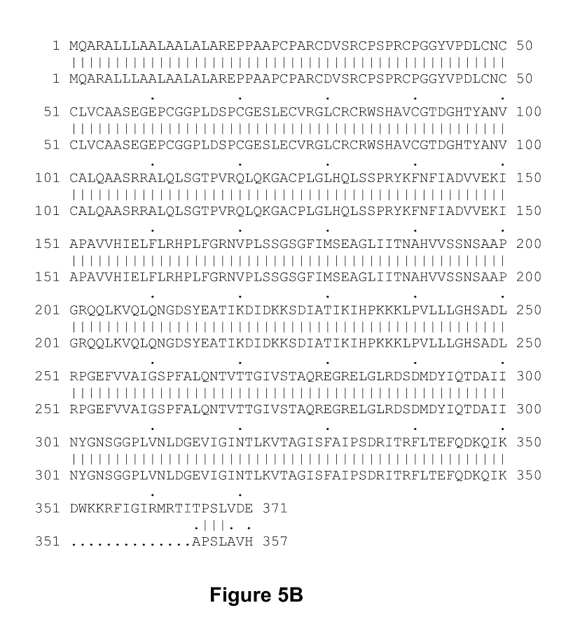

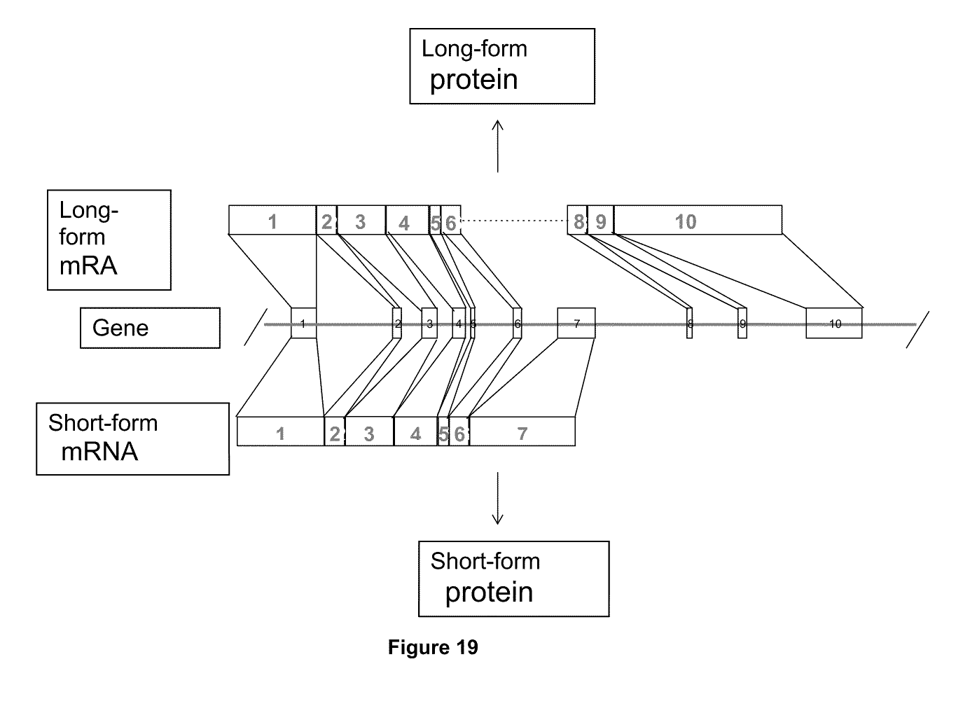

These two isoforms are identical at the cDNA level, up to nt 1243 on the longer isoform and nt 1158 on the short isoform, as shown in FIG. 5A. At the protein level, the short isoform is substantially smaller than the longer one, but otherwise they are exactly the same except for the few amino acids at the C-terminal ends, as shown in FIG. 5B. It is considered that the two isoforms are derived from alternative splicing of the same primary RNA molecule. A proposed molecular mechanism for the generation of long and short isoforms of PRSP protein due to alternative splicing of the pre-mRNA in the mouse and human is shown in FIG. 19.

When compared to the mouse sequences described herein, the human longer isoform is 79% identical at the cDNA level and 93% identical at the protein level to the mouse longer isoform, and the short isoform is 87% identical at the cDNA level and 92% identical at the protein level to the mouse short isoform. Therefore these human sequences are the true counterparts of the mouse sequences. However, when compared to the human HtrA sequences, the longer isoform is only 67% identical at the cDNA level and 61% identical at the protein level to the human HtrA, and the shorter form is 71% identical at the cDNA level and 65% identical at the protein level to the human HtrA. Therefore these newly cloned human sequences are quite different from those of the human HtrA.

Subsequently it has been determined that the human PRSP protein sequence provided as SEQ ID 33 is substantially homologous to SEQ ID NO: 3 in WO 00/39149 to Millenium Pharmaceuticals, Inc.). SEQ ID NO: 3 was not previously suggested to be involved in embryo implantation.

As observed for the mouse sequences, the 16 cysteine residue IGF-binding motif and the serine proteases motifs GNSGGPL and TNAHV are also present in the human sequences; therefore these two human isoforms also represent serine proteases with IGF-binding domains.

### EXAMPLE 5

### Identification of Two Isoforms of the Mouse Enzyme

The possible existence of similar long and short isoforms to those demonstrated for the human enzyme in Example 4 was examined in the mouse. Sequence comparison between the human and mouse indicated that the mouse cDNA sequence shown in FIG. 2 probably represents the longer isoform. On the assumption that the splicing characteristic in the mouse would be the same as that in the human, a possible splice site was located on the mouse cDNA sequence at around nt 1185 (as shown in FIG. 2).

A forward primer (5′ GGC ATC AAC ACG CTC AA 3′ (SEQ ID NO:35), nt 1096-1112 of SEQ ID NO:26) upstream from this possible splicing site was designed, and 3′ RACE was performed using this forward primer plus an Oligo d(T)-anchor primer (5′ GAC CAC GCG TAT CGA TGT CGA CTT TTT TTT TTT TTT TTV 3′ (SEQ ID NO:36), available from the 5′/3′ RACE kit) and mRNA was isolated from day 10.5 placenta. Surprisingly, two bands with the sizes expected for the presumed two isoforms were indeed amplified (data not shown); however, the intensity of the shorter isoform band was much lower than that of the longer isoform one. This indicated that in the mouse, in addition to the cDNA cloned in Example 2 (SEQ ID NO:26), another isoform differing at the 3′ end did exist in the mouse, and that the expression level of the longer isoform may be much higher than that of the short isoform.

These two 3′ RACE products were subsequently cloned and sequenced, and it was confirmed that the longer one represented the 3′ end of the cDNA sequence cloned in Example 2 (SEQ ID NO:26), and the shorter one represented the 3′ end of a cDNA encoding another isoform.

In order to clone the full cDNA sequence of this shorter isoform and to confirm that the two isoforms are different only at the 3′ end, a mouse uterine cDNA library specific to the pregnant uterus was constructed using mouse uterine tissues obtained on day 4.5 and 5.5 of pregnancy. Poly-(A)⁺ mRNA was isolated from total RNA of day 4.5-5.5 pregnant uterus using the PolyATract mRNA Isolation System (Promega). The resulting mRNA (5 μg) was used to construct a mouse cDNA library specific to the pregnant mouse uterus, using the ZAP Express cDNA synthesis and ZAP Express cDNA Gigapack III Gold Cloning Kit (Stratagene, La Jolla, Calif., USA). This cDNA library was screened using a short isoform-specific sequence (476 bp) derived from the 3′RACE cloning as a probe. The sequence of the probe is set out in Table 3 (SEQ ID NO:37).

TABLE 3

A 476 bp probe used as short-isoform specific sequence
(SEQ ID NO: 37)

| | |
|---|---|
| 1 | CCATGAAGAA CTGCAACCGA GGAGCCTCGT TCTGTTCCAA GTGGCCCTAT |
| 51 | ATGAAGATGA CAGGAGCAGG CAGAGCCTGT CCCTTCCAGG AATCCGAGAC |
| 101 | ACCTTCTGGT GAATAGTGGG AACTAGCTGC CTTTTCTCTT GGCCGGTAGG |
| 151 | AAGCTCAGAA CTAGACCAGG GTTCCTAGAC CATTGGTAGC CTTGGCTCTT |
| 201 | TGTCTAGTGG CCAGGGCTTT CCAGTTTAGC TTGTTTATGG GGTCGGAACA |
| 251 | CCACCCACAT ACACTGGCCT ATGGGTGATT ACTGTGCTGG AAATGGGCCA |
| 301 | GCGGCCTTTT GTCCCCTAGC TGTCTCATCT TTTCTCAGAC AAGAAGTCCC |
| 351 | CGGGGCAGGA TCTGCTCCTC TGTGGCAGAG CAACTATCCT AGTCACAGTG |
| 401 | ACCTGGTCAC TCAGCCTGGG CTCTGCGGAA ATGCTCACAC CCATCCCAGA |
| 451 | GTTATGTTAT CACCCAAGGA CAGTGC |

Several clones were analysed, and the full length cDNA sequence was obtained; this is presented in FIG. 6A (SEQ ID NO:38). This shorter isoform cDNA contained 1897 nt compared to 2450 nt for the longer isoform; the two sequences are exactly the same until nt 1195, but beyond this point they are very different, indicating that they are indeed derived from alternative splicing. In the open reading frame, the short isoform cDNA contained a stop codon TGA at nt 1216-1218 (FIG. 6A) instead of nt 1504-1506 in the long isoform (FIG. 2); therefore the short isoform cDNA codes for a protein of 363 amino acids, instead of 459 for the long isoform cDNA. The protein sequence is shown in FIG. 6B (SEQ ID NO:39).

However, all the characteristics such as the cysteine residues, active serine protease sites etc described earlier for the longer isoform are presented by the short isoform (FIG. 6C), indicating that although the shorter protein is still an active serine protease, its function or sub-cellular location may differ. The difference between the two may also lie in the substrate specificity or sub-cellular localisation.

### EXAMPLE 6

### Determination of mRNA Expression in the Mouse Uterus During Early Pregnancy

After determination of the full cDNA sequence encoding the novel protein, additional Northern analyses were performed to systematically determine the expression pattern of this gene in the uterus in relation to the time of implantation and early pregnancy. A 785 by cDNA sequence (SEQ ID NO:40; nt 76-860 of the longer isoform cDNA, shown in FIG. 2) representing the common region of the two isoform cDNAs was used as a probe to detect both isoforms on the same gel; the sequence of this probe is set out in Table 4.

TABLE 4

A 785 bp sequence common to both mouse isoforms
(SEQ ID NO: 40) used as a probe

| | |
|---|---|
| 1 | GCGGTTCGGG CCTCGGTATC CCCGCGGGTC TTGCGCCGCC GCCTCTCCGC |
| 51 | GATGCAGGCG CGCGCGCTGC TCCCCGCCAC GCTGGCCATT CTGGCCACGC |
| 101 | TGGCTGTGTT GGCTCTGGCC CGGGAGCCCC CAGCGGCTCC GTGTCCTGCG |
| 151 | CGCTGCGACG TGTCGCGCTG TCCGAGCCCT CGCTGCCCTG GGGGCTATGT |
| 201 | GCCTGACCTC TGCAACTGCT GCCTGGTGTG CGCTGCCAGC GAGGGCGAGC |
| 251 | CCTGCGGCCG CCCCCTGGAC TCTCCGTGCG GGGACAGTCT GGAGTGCGTG |
| 301 | CGCGGCGTGT GCCGCTGCCG TTGGACCCAC ACTGTGTGTG GCACAGACGG |
| 351 | GCATACTTAT GCCGACGTGT GCGCGCTGCA GGCCGCCAGC CGTCGTGCGT |
| 401 | TGCAGGTCTC CGGGACTCCA GTGCGCCAGC TGCAGAAGGG TGCCTGTCCC |
| 451 | TCTGGTCTCC ACCAGCTGAC CAGTCCGCGG TACAAGTTCA ACTTCATCGC |
| 501 | CGATGTGGTG GAGAAGATTG CGCCAGCTGT GGTCCACATA GAGCTCTTTC |
| 551 | TGAGACACCC CCTGTTTGGC CGGAATGTGC CGCTGTCCAG TGGCTCGGGC |
| 601 | TTCATCATGT CAGAAGCCGG TTTGATCGTC ACCAACGCCC ACGTGGTCTC |
| 651 | CAGCTCCAGC ACTGCCTCCG GCCGGCAGCA GCTGAAGGTG CAGCTGCAGA |
| 701 | ATGGGGATGC CTATGAGGCC ACCATCCAGG ACATCGACAA GAAGTCGGAC |
| 751 | ATTGCCACGA TTGTAATCCA CCCCAAGAAA AAGCT |

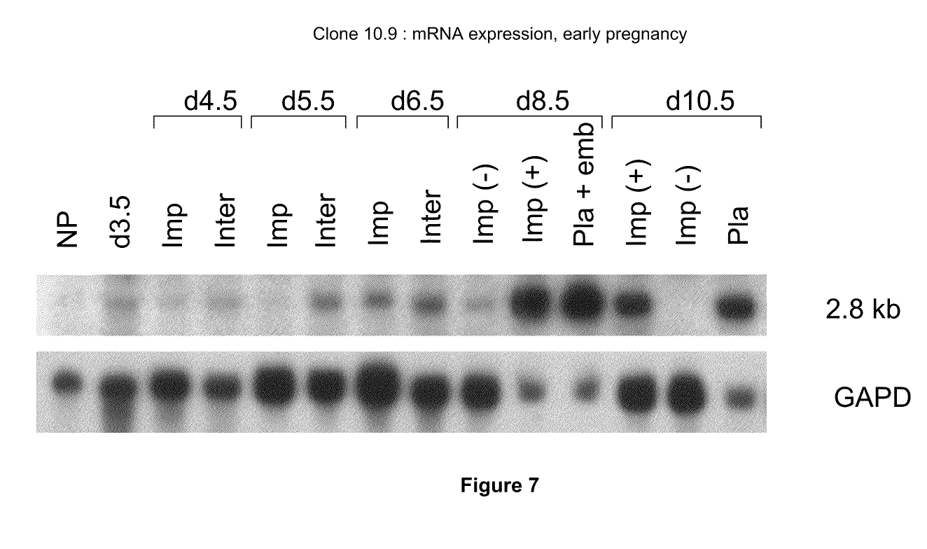

Total RNA from the uterus of non-pregnant mice (estrus) and pregnant mice at the initial stage of implantation (day 4.5 of pregnancy) through to fully established implantation and placentation (day 10.5 of pregnancy) was analysed, and the results are shown in FIG. 7. Very low expression was observed in non-pregnant mice, and a marginally higher level was seen on day 3.5 of pregnancy. Around days 4.5 and 5.5 of pregnancy the expression was still quite low, but it was relatively higher in the interimplantation sites compared to the implantation sites. Around day 6.5 of pregnancy, similar levels of expression were detected in both implantation and interimplantation sites. Beyond day 6.5, a dramatic up-regulation of this gene occurred, and by day 8.5-10.5, the mRNA level was several fold higher than that detected in the interimplantation sites on day 4.5-5.5. Dissection of the maternal-fetal unit on day 10.5 revealed that this up-regulation mainly occurred in the placental tissues. Interestingly, the band pattern detected by these analyses showed that only the longer isoform was expressed, and that the expression of the short form was at a level below the detection sensitivity of the Northern blot technique.

Northern blotting studies using human tissues sampled at different stages of the endometrial cycle and in early pregnant tissues showed the expression of the novel protease, and of HtrA, with different patterns of expression being observed for these two enzymes. A 384 bp probe was used for this experiment, and its sequence (SEQ ID NO:41) is set out in Table 5.

TABLE 5
A 384 bp human sequence (SEQ ID NO: 41) used as a probe

| | |
|---|---|
| 1 | AAAGCCATCA CCAAGAAGAA GTATATTGGT ATCCGAATGA TGTCACTCAC |
| 51 | GTCCAGCAAA GCCAAAGAGC TGAAGGACCG GCACCGGGAC TTCCCAGACG |
| 101 | TGATCTCAGG AGCGTATATA ATTGAAGTAA TTCCTGATAC CCCAGCAGAA |
| 151 | GCTGGTGGTC TCAAGGAAAA CGACGTCATA ATCAGCATCA ATGGACAGTC |
| 201 | CGTGGTCTCC GCCAATGATG TCAGCGACGT CATTAAAAGG GAAAGCACCC |
| 251 | TGAACATGGT GGTCCGCAGG GGTAATGAAG ATATCATGAT CACAGTGATT |
| 301 | CCCGAAGAAA TTGACCCATA GGCAGAGGCA TGAGCTGGAC TTCATGTTTC |
| 351 | CCTCAAAGAC TCTCCCGTGG ATGACGGATG AGGA |

Reverse transcriptase polymerase chain reaction (RT-PCR) also detected the expression of HtrA and of both isoforms of PRSP in the endometrium across the human endometrial cycle, in early and late human pregnant tissues (placenta and decidua), in pre- and post-menopausal ovary, ovary, heart and skeletal muscle, as shown in FIG. 11.

### EXAMPLE 7

### Tissue Distribution of mRNA Expression

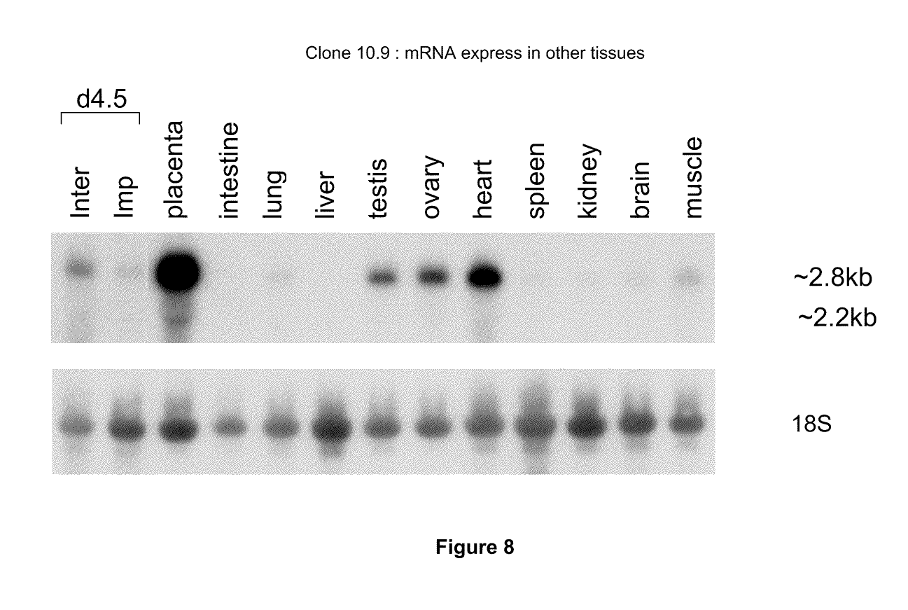

Multi-tissue Northern analysis was performed to investigate the tissue distribution of mRNA expression of the protease. As shown in FIG. 8, the protease was not widely expressed in mice. When an equal amount of total RNA was compared, the day 10.5 placenta showed the highest level of expression; this placental level is several fold higher than that seen in the interimplantation sites on day 4.5 of pregnancy. Of the 12 tissues tested, apart from the uterus, the testis, ovary and heart had moderate expression, while muscle and lung had low expression. On this Northern blot a faint band representing the short isoform was detected in the placenta, but the level was very low.

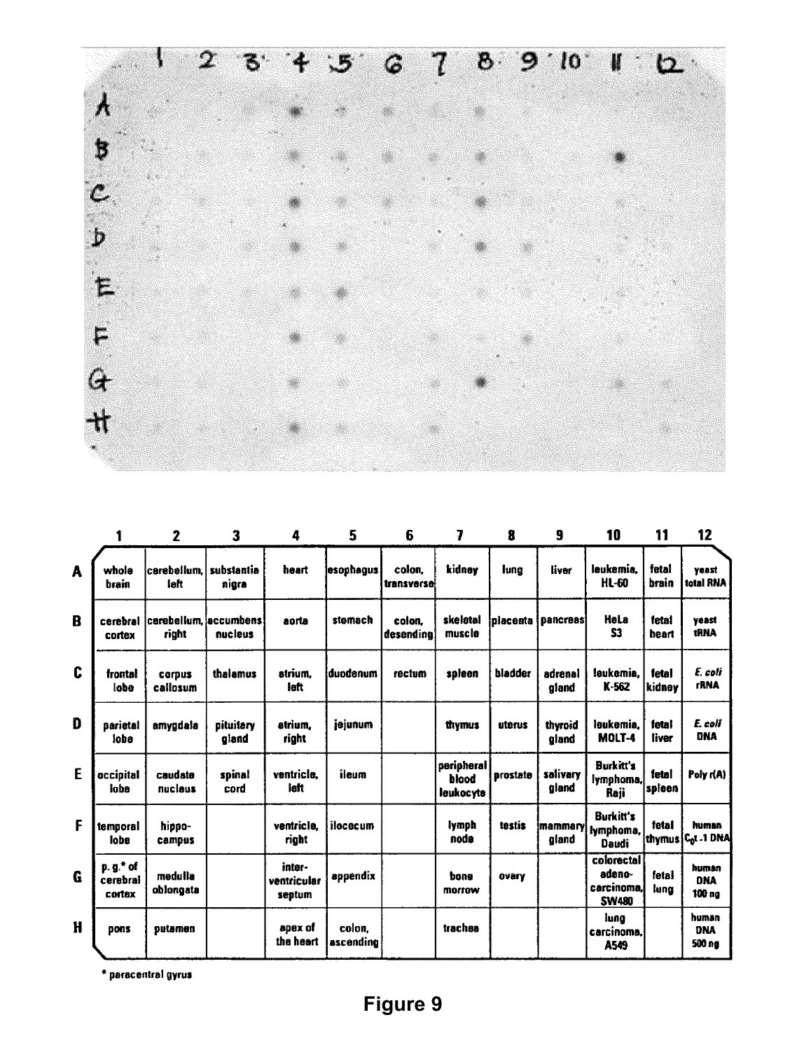

The human MTE array was probed with a sequence common to both isoforms, using the sequence used in Example 6, Table 5 (SEQ ID NO:41). The results, shown in FIG. 9, indicated that heart, ovary, and uterus all expressed the novel protease. However, the expression pattern was quite different when HtrA was probed on the same MTE, indicating that these two enzymes are distributed quite differently.

Probing a commercial Northern blot (Clontech, Palo Alto, Calif.) with the same probe (SEQ ID NO:41) also identified the expression of the two isoforms in human placenta, heart and other tissues, including lung, liver, kidney and skeletal muscle tissue.

### EXAMPLE 8

### Southern Analysis of Mouse Genomic DNA

Mouse genomic DNA was isolated from the uterus and the kidney, and the DNA was subjected to Southern analysis. Total genomic DNA was isolated from non-pregnant uterus and the kidney using the DNeasy Tissue Kit (Qiagen). A total amount of 10 μg was digested separately with an excess of several restriction endonucleases (Tag 1, Hind III, ECoRI BamHI) at 37° C. for 14 hours and fractionated on 0.8% agarose gel. The DNA was then blotted on to positively-charged nylon membranes (Hybond-N, Amersham) using the standard Southern blotting procedure, and probed with radiolabelled cDNA as described for the Northern analysis.

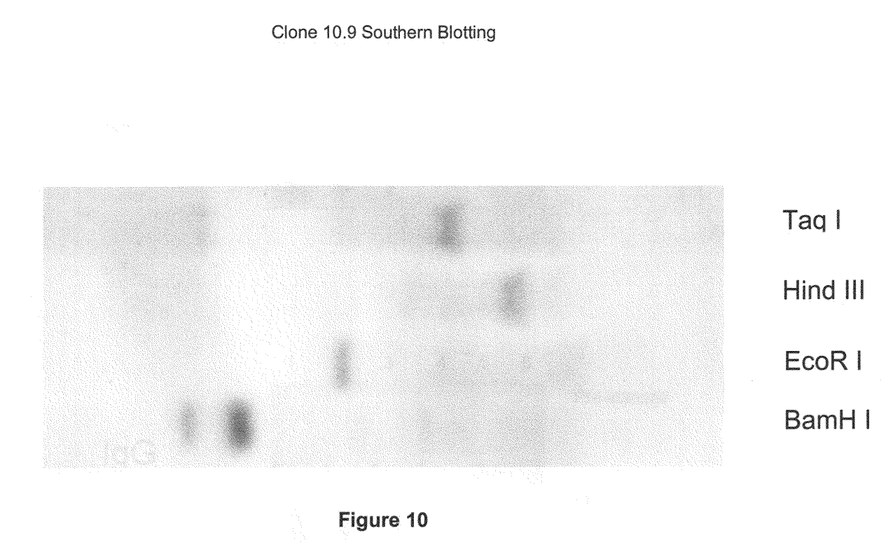

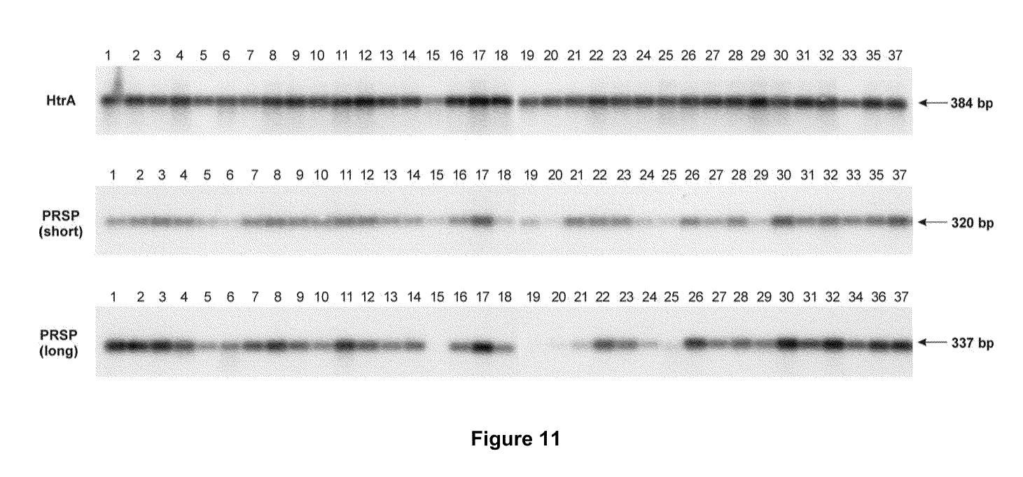

Similar results were obtained for the two tissue types; thus only the result with the uterus will be discussed. FIG. 10 shows the results of Southern analysis of mouse genomic DNA from non-pregnant uterus digested separately with TaqI, HindIII, ECoRI and BamHI and probed with a radiolabelled cDNA probe representing both isoforms (SEQ ID NO:40). In all cases, the digestion pattern was quite simple, indicating that this gene is represented by a single copy in the genome.

### EXAMPLE 9

### Detection of PRSP and HtrA in Cycling and Pregnant Human Endometrium

Semiquantitative Reverse transcriptase polymerase chain reaction (RT-PCR) Southern blot analysis was performed to investigate the mRNA expression of PRSP (long and short forms) and HtrA in human endometrium during the menstrual cycle and early pregnancy. Samples of human heart and skeletal muscle were used as positive controls.

Primers used for long form PRSP were:

| | |
|---|---|
| Upper primer: | |
| 5′ ATG CGG ACG ATC ACA CCA AG 3′ | SEQ ID NO: 42 |
| Lower Primer: | |
| 5′ CGC TGC CCT CCG TTG TCT G 3′ | SEQ ID NO: 43 |

An expected band of 337 by was detected.

Primers used for the short form PRSP were:

| | |
|---|---|
| Upper primer: | |
| 5′ GAG GGC TGG TCA CAT GAA GA 3′ | SEQ ID NO: 44 |
| Lower Primer: | |
| 5′ GCT CCG CTA ATT TCC AGT 3′ | SEQ ID NO: 45 |

An expected band of 320 by was detected.

Primers used for HtrA were:

| | |
|---|---|
| Upper primer: | |
| 5′ AAA GCC ATC ACC AAG AAG AAG TAT 3′ | SEQ ID NO: 46 |
| Lower Primer: | |
| 5′ TCC TCA TCC GTC ATC CAC 3′ | SEQ ID NO: 47 |

An expected band of 384 by was detected.

The results are shown in FIG. 11. Both the short and long form mRNA of PRSP were detected in the human endometrium during the menstrual cycle. They were also expressed in the first trimester decidua and placenta. HtrA was also detected in all samples. However, the expression pattern of PRSP was different from that of HtrA.

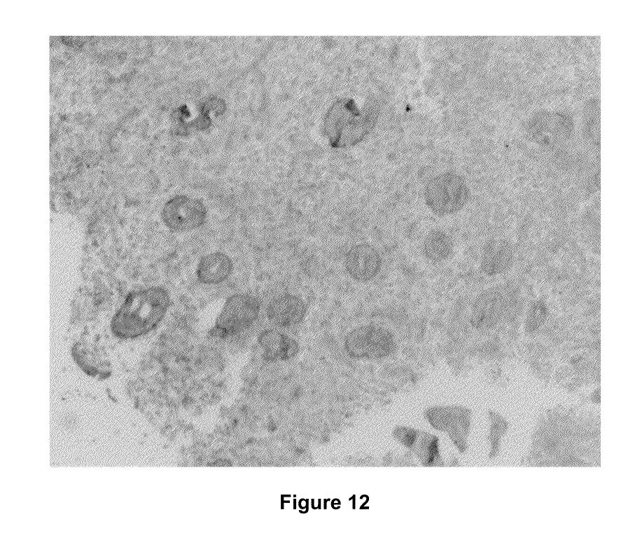

For studies of human tissues, a 396 by probe for a sequence (SEQ ID NO: 50) common to both isoforms of human PRSP mRNA was used for in situ hybridization studies. FIG. 12 shows the results of in situ hybridization detection of PRSP mRNA in cycling human endometrium at day 9 of the menstrual cycle.

### EXAMPLE 10

### Antibodies Directed against the Novel Enzyme

Antibodies against the novel protease and against HtrA were produced using conventional methods. Sheep were immunized with peptides derived from the mouse protein. The following peptides were synthesised using conventional solid phase synthetic methods, and used as antigens:

(SEQ ID NO: 51)

1. Amino acids 133-142; sequence PSGLHQLTSP

(SEQ ID NO: 52)

2. Amino acid 116-126; sequence ALQVSGTPVRQ

(SEQ ID NO: 53)

3. A sequence common to both isoforms; amino acids
313-324 sequence GPLVNLDGEVIG

Peptides 1-3 are from the mouse PRSP sequence, which is highly homologous to that of the human PRSP protein. It will be appreciated that other peptides could also be used.

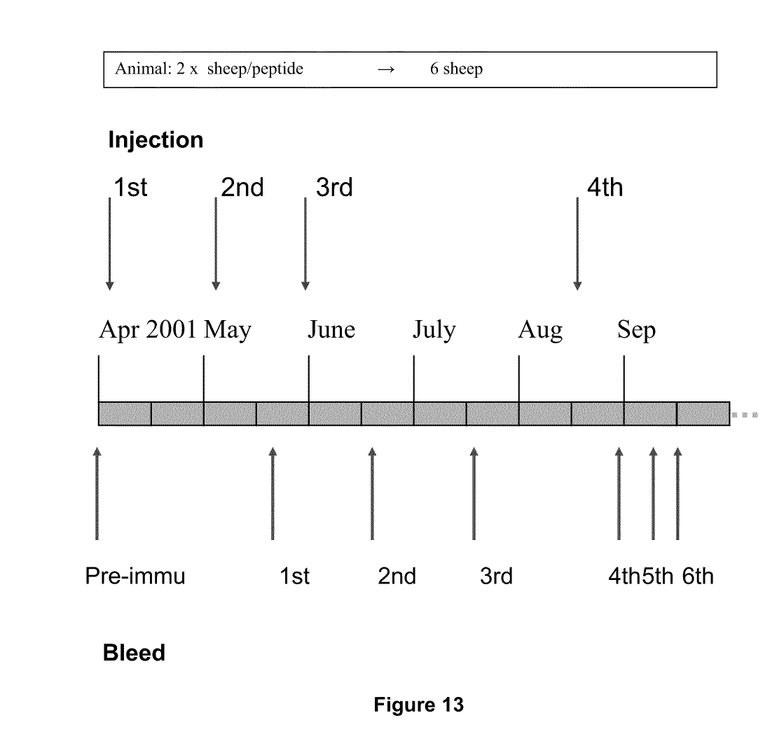

An additional cysteine was added at the C-terminal end of each peptide to allow conjugation. The peptides were conjugated to diptheria toxoid, and the conjugated protein homogenized in an adjuvant comprising QuilA/DEAE-Dextran/Montanide 888, as described in Prowse (2000) prior to each injection. Sheep were immunized with the material at 4 weekly intervals for 3 or more injections, and bled between 1 and 2 weeks following the second and subsequent injections. The immunisation scheme is illustrated in FIG. 13.

The presence of anti-PRSP antibodies in the sheep serum following immunisation against specific peptides of PRSP was examined by dot blot. Peptides were dotted and dried onto Hybond-P™ membranes (Amersham Life Sciences). After blocking the non-specific binding sites with 5% (w/v) skim milk powder in TBS with 0.1% Tween 20 for 1 h, blots were incubated for 1 h at room temperature with a 1:2,000 dilution of serum. Blots were then incubated with horseradish peroxidase-labelled donkey anti goat/sheep IgG (Silenus) diluted to 1:20,000. All antibody dilutions were in 5% (w/v) skim milk powder in TBS with 0.1% Tween 20. Blots were developed by chemiluminescence (ECL Plus system, Amersham). As a negative control, pre-immune serum from the same animal was used and a non-related peptide was tested on each blot.

In addition, total IgG was prepared by ammonium sulphate precipitation following capryllic acid treatment of whole serum. The presence of specific antibodies in the total IgG was also examined by dot blot.

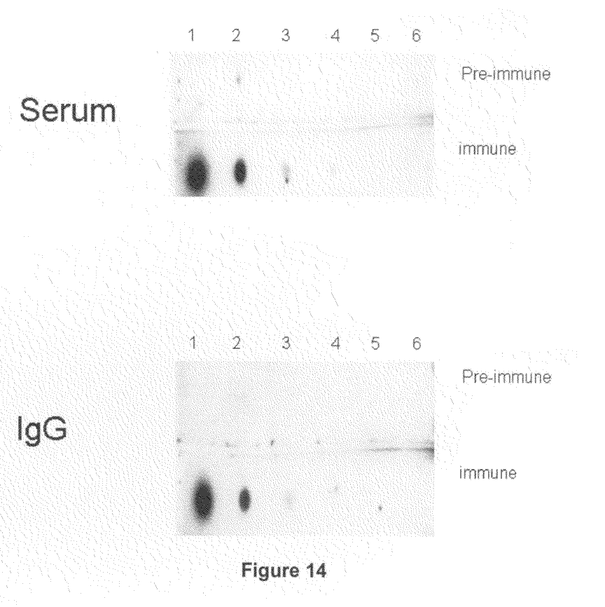

Results for antibodies raised against peptide (2) aa 116-126 are shown in FIG. 14. The presence of specific antibodies in both the whole sheep serum and in total IgG prepared from the serum was demonstrated by specific reactions with the spots containing the specific peptides of PRSP. The specificity of the antibodies was further demonstrated by the following evidence:
(1) no reaction was detected with pre-immune serum or total IgG (at the same concentration as the antibody) prepared from the pre-immune serum;
(2) no reaction was detected on spots containing irrelevant peptides of equivalent size;
(3) a dose-dependent reaction was detected with serial dilution of the specific peptides.

### EXAMPLE 11

### Western Blotting Studies of Human and Mouse Tissues

Specific IgG was further purified from the total IgG (ammonium sulphate precipitate) by affinity purification using a HiTrap affinity column (Amersham Pharmacia Biotech). The expression of PRSP protein in the mouse and human uterus was detected with the affinity purified antibodies by Western blot. Proteins were extracted from one sample of human endometrium on day 25 of the menstrual cycle, one sample of non-pregnant mouse uterus and one mouse placenta on day 10.5 of pregnancy. Weighed tissue was homogenised in 6% SDS, 0.14M Tris (pH 6.8) and 22.4% glycerol (2 ml per 100 mg of tissue) with a protease inhibitor cocktail (Calbiochem, Croyden, Australia; 5 μl per 100 mg of tissue). The homogenate was then passed sequentially through 21, 23 and 25 gauge needles followed by centrifugation at 14,000 g at 4° C. for 15 min. 15 μg of total protein from each supernatant, together with molecular weight markers (Kaleidoscope prestained standards; Biorad) were subjected to SDS-PAGE on a 12% gel under reducing conditions. The proteins were transferred to Hybond-P™ membranes (Amersham Life Sciences, Sydney). After blocking non-specific binding sites with 5% (w/v) skim milk powder in TBS with 0.1% Tween 20 for 1 h, blots were incubated for 1 h at room temperature with 100 μg/ml of affinity-purified IgG in 5% (w/v) skim milk powder in TBS with 0.1% (v/v) Tween 20. Blots were then incubated with horseradish peroxidase-labelled donkey anti goat/sheep IgG (Silenus) diluted to 1:20,000 and developed by chemiluminescence (ECL Plus system, Amersham). The presence of PRSP protein in the serum of pregnant women was also detected by Western blot analysis of 2 μl serum following TCA precipitation.

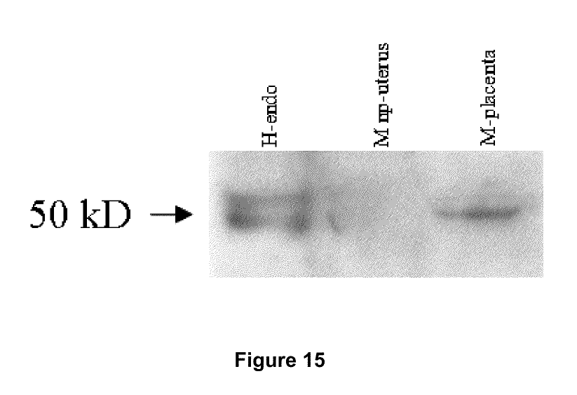

As shown in FIG. 15, Western blot analysis detected the expression of PRSP protein in human endometrium and mouse uterus, indicating the presence of PRSP protein in tissues where its mRNA was detected. The bands detected correlated well with the anticipated size of the protein in both the human and mouse. In the human, two bands corresponding to the two isoforms of PRSP were detected, indicating the expression of both isoforms of PRSP protein. This agrees very well with the mRNA data, where both the long and short form of PRSP mRNA was detected in the human endometrium. The polyclonal antibody raised against peptide 2 (mouse sequence ALQVSGTPVRQ (SEQ ID NO: 52) recognized mouse PRSP as well as both isoforms of human PRSP. In the mouse, only one form of PRSP and much higher expression was detected in the placenta on day 10 of pregnancy, compared with the non-pregnant uterus. This is consistent with the mRNA expression data where an abundant level of only the long form of PRSP was detected in the pregnant uterus.

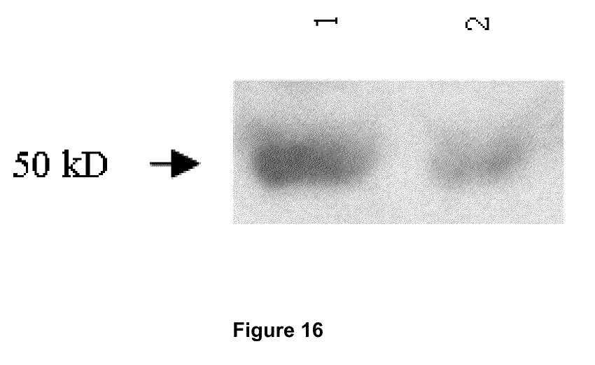

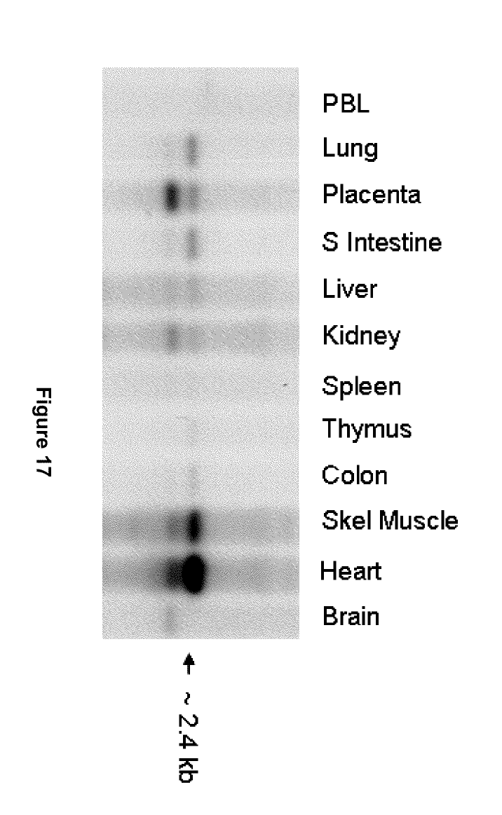

As shown in FIG. 16, Western blot analysis using the polyclonal antibody raised to peptide 2 (SEQ ID NO: 52) also detected PRSP protein in the serum of pregnant women. The origin of this protein is considered to be the developing placenta during pregnancy. Thus the maternal serum profile of PRSP during pregnancy may be associated with placental development and function, and it is anticipated that the serum profile of PRSP might provide a marker for predicting placenta-related complications of pregnancy.

### EXAMPLE 12

### Northern Analysis of PRSP in a Range of Human Tissues

A human multi-organ Northern blot (Clontech) containing 1 μg of poly A⁺ RNA isolated from each of a range of human tissues was probed with a 457 by cDNA sequence representing the common region of the two isoform cDNAs of human PRSP. The sequence of this probe is set out in Table 6.

TABLE 6

The 457 bp sequence (SEQ ID NO: 54) common to both
isoforms of human PRSP mRNA used as a probe for
Northern blotting

| | |
|---|---|
| 1 | GCGGTTCTGG CTTCATCATG TCAGAGGCCG GCCTGATCAT CACCAATGCC |
| 51 | CACGTGGTGT CCAGCAACAG TGCTGCCCCG GGCAGGCAGC AGCTCAAGGT |
| 101 | GCAGCTACAG AATGGGGACT CCTATGAGGC CACCATCAAA GACATCGACA |
| 151 | AGAAGTCGGA CATTGCCACC ATCAAGATCC ATCCCAAGAA AAAGCTCCCT |
| 201 | GTGTTGTTGC TGGGTCACTC GGCCGACCTG CGGCCTGGGG AGTTTGTGGT |
| 251 | GGCCATCGGC AGTCCCTTCG CCCTACAGAA CACAGTGACA ACGGGCATCG |
| 301 | TCAGCACTGC CCAGCGGGAG GGCAGGGAGC TGGGCCTCCG GGACTCCGAC |
| 351 | ATGGACTACA TCCAGACGGA TGCCATCATC AACTACGGGA ACTCCGGGGG |
| 401 | ACCACTGGTG AACCTGGATG GCGAGGTCAT TGGCATCAAC ACGCTCAAGG |
| 451 | TCACGGC |

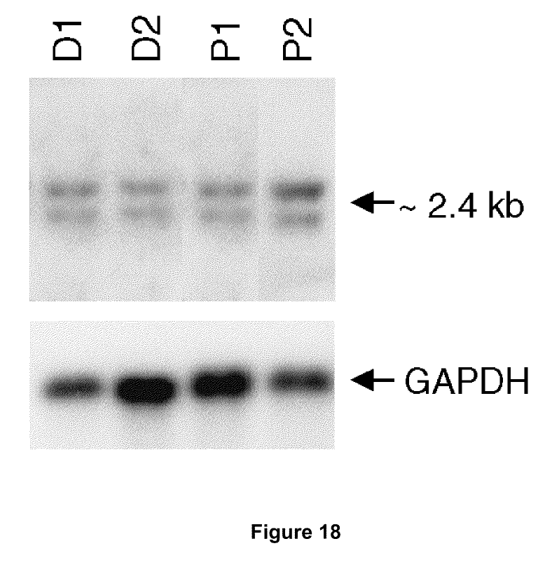

As shown in FIGS. 17 and 18 strong positive signals were detected in the heart, skeletal muscle and placenta. Lung, small intestine and kidney showed low expression while liver, thymus, colon and brain showed minimal expression. No expression was detected in the peripheral blood leukocytes and the spleen. The transcript sizes detected were around 2.4 kb. It is very interesting to note that two bands, representing the two isoforms of PRSP mRNA, were detected in the placenta, heart, skeletal muscle and kidney. The long form was predominant in the lung and small intestine, and the short form was predominant in the brain.

### EXAMPLE 13

### Northern Analysis of PRSP mRNA in the First Trimester Placenta and Decidua

Total RNA was isolated from first trimester pregnant human decidua and placenta, and the expression of PRSP mRNA was analysed by Northern blotting. The same 457 by cDNA sequence as that used in Example 12, representing the common region of the two isoform cDNAs of human PRSP, was used. The results are shown in FIG. 19. Strong positive signals were detected in both the placenta and decidua. Two bands of approximately 2.4 kb, representing the two isoforms of PRSP mRNA, were detected in all samples.

### EXAMPLE 14

Mice were genetically modified to render them null for the mouse gene encoding a long and short form serine protease (SEQ ID NO. 26 and 38) whose expression corresponds to proteins (SEQ ID NO. 27 and 39). Wild-type (+/+), heterozygous (+/−) and homozygous (−/−) female mice (approx 8 weeks old) were mated with males of the same strain. Pregnant mice were killed at day 18 of pregnancy (E18, the day before birth) and the fetuses and placentas weighed.

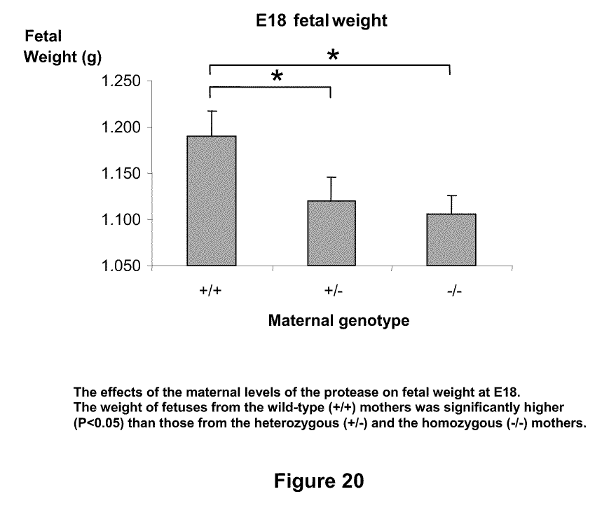

Overall, there was a significant decrease in fetal weight on E18 (P<0.05) in the fetuses derived from the −/− or +/− mothers compared with the +/+ mothers (FIG. 20), while all three types of mothers had similar numbers of viable fetuses on E18.

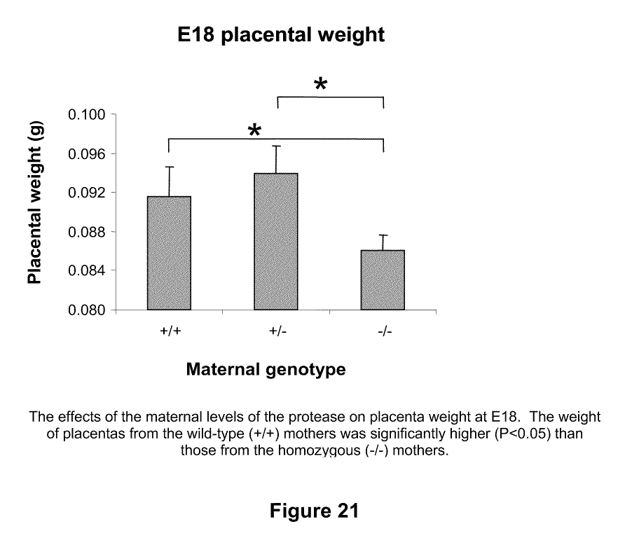

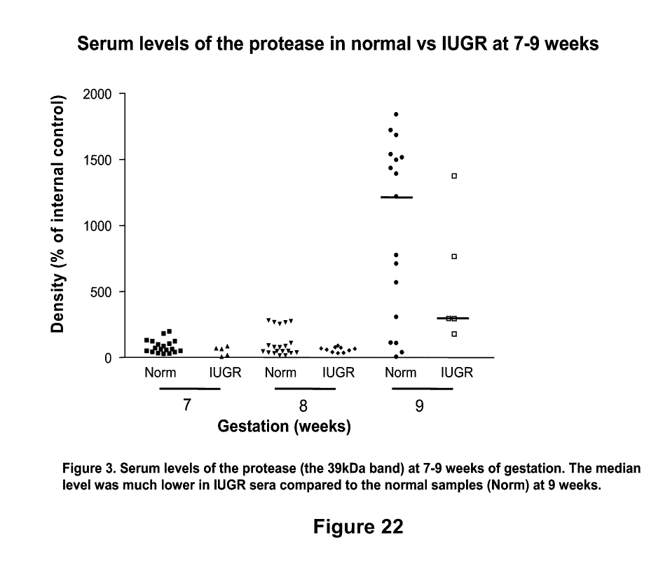

There was also a significant decrease in placental weight on E18 (P<0.05) in the −/− mothers compared with the +/− mothers (FIG. 21).

Accordingly, deficiency of the PRSP serine protease shown above to be upregulated at the site of embryo implantation in mothers, results in low birth weight fetuses and small placentas in mice. This supports the hypothesis that the protease is critical for placental development and function.

The null mouse provides a further model to study infertility conditions.

### EXAMPLE 15

Serum samples were taken from women between 7-9 weeks of pregnancy. All of these women delivered full-term babies without any obvious pregnancy complications. The women were separated into two groups according to the birth weight of their babies at term: a) women gave birth to babies of normal birth weight (>3.3 kg) and b) women gave birth to smaller babies (2.1-2.7 kg).

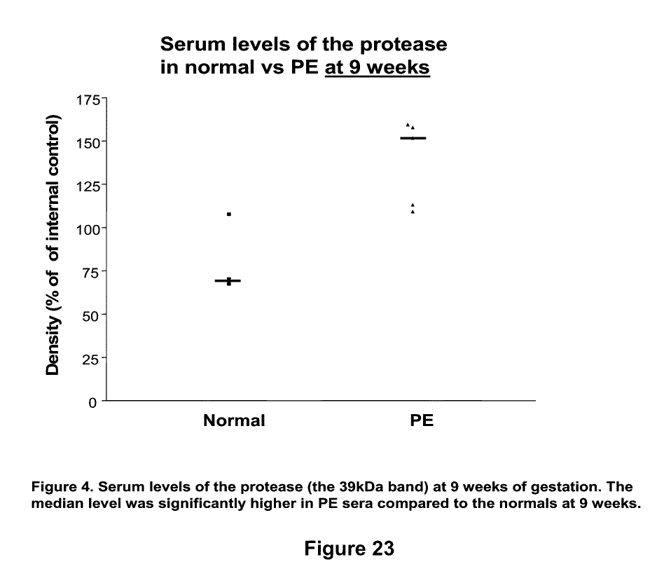

Sera were subjected to Western blot analysis using an antibody specific for PRSP (raised against SEQ ID NO: 52). A band at 39 kDa was detected in all the sera; the density of the bands was lower at 7 and 8 weeks of pregnancy and increased dramatically at 9 weeks in both groups (FIG. 22). No difference in density was seen between the two groups at 7 weeks, at 8 weeks the density of the band was slightly lower in women who delivered smaller babies and the density of the band was much lower at 9 weeks of pregnancy in women who delivered smaller babies (FIG. 22) compared to control at 9 weeks.

These data strongly suggest that the lower levels of the protease in the maternal blood during first trimester are closely associated with higher risks of delivering a low-birth-weight baby at term. This supports the hypothesis that measurement of the expression of the serine protease between 8-15 weeks of pregnancy is diagnostic of IUGR.

### EXAMPLE 16

Serum samples were taken from women between 7-15 weeks of pregnancy and subsequently separated into two groups. a) women who underwent normal pregnancy and gave birth to healthy normal babies or b) women who subsequently developed pre-eclampsia (PE).

Sera were subjected to Western blot analysis using the same antibody specific for PRSP as described in Example 15. A PRSP band around 39 kDa was detected in all the sera. At 9 weeks of pregnancy, the density of the PRSP band was significantly higher (P<0.05) in women who subsequently developed PE compared to the controls (FIG. 23).

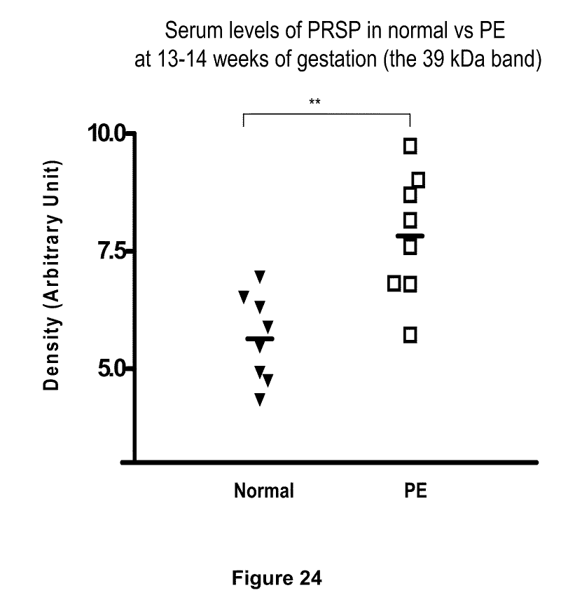

The density of the 39 kDa PRSP band, the dominant band of PRSP, in the sera at 13-14 weeks of pregnancy are significantly higher in women who subsequently developed PE compared to the controls (FIG. 24).

The blood level of PRSP in first-trimester of pregnancy is different between women who subsequently develop or do not develop PE. This supports the hypothesis that measurement of the protease in the maternal blood during early stages of pregnancy may provide an early diagnostic test for PE.

These data provide strong evidence that monitoring PRSP in the maternal blood during early pregnancy may identify women who have higher risks of developing PE at later stages of pregnancy.

It is important to note that the measuring of the blood level of the protease at 9 weeks of gestation can differentiate between IUGR and PE conditions.

It is envisaged that PRSP levels will continue to distinguish IUGR and PE throughout early stage pregnancy.

### EXAMPLE 17

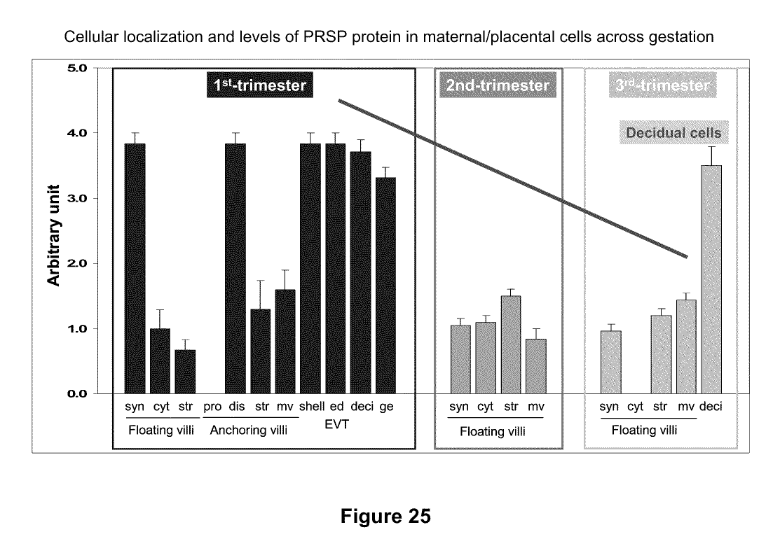

The cellular localization and expression levels of PRSP in the human placenta were determined by immuno-histochemistry on formalin fixed paraffin embedded samples using the same antibody as described above at 8-10 weeks (1^{st} trimester), 2^{nd} trimester and at term. The localization and expression levels in the following cells was determined and shown in FIG. 25:

Syn, syncytiotrophoblast; cyt, cytotrophoblast; str, stroma; pro, proximal region of the anchoring villi; dis, distal region of the anchoring villi; my, microvilli on the cells; shell, trophoblast shell; ed, endovascular trophoblast; deci, decidual cells; ge, glandular epithelial cell of the endometrium. The bar represents the overall decrease in expression levels of PRSP in the placenta as pregnancy proceeds.

In the 1^{st} trimester placenta, PRSP was localized in floating villi, anchoring villi and extravillous trophoblasts. In the floating villi, PRSP was detected most strongly in the syncytiotrophoblast, whereas the levels in the cytotrophoblast and stroma were much lower. In the anchoring villi, PRSP was detected strongly in the distal region of the columns. In the extravillous trophoblasts, the trophoblast shell and endovascular trophoblasts were strongly positive for PRSP. In addition, the decidual cells and the glandular epithelium of the uterus were also strongly positive for PRSP (FIG. 25).

In the 2^{nd} trimester and term placenta, PRSP was detected mainly in the syncytiotrophoblast and the fetal capillary. The decidual cells at term were also strongly positive for PRSP.

The overall levels of PRSP protein in the placenta were much higher in the 1^{st} trimester of pregnancy.

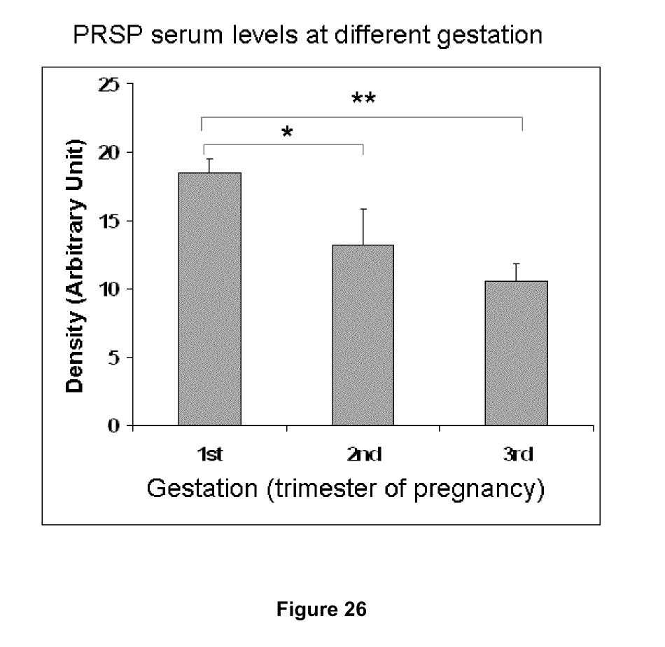

The serum levels of PRSP were also determined at different times of normal pregnancy: 7-10 weeks (1^{st} trimester), (2^{nd} trimester) and term. The method of Western blotting was the same as the previous examples. The serum PRSP levels were highest in the 1^{st} trimester (FIG. 26). This indicates that the dynamic expression of PRSP in the placenta across gestation (FIG. 25) was reflected by a similar trend of change in the maternal blood. This supports the hypothesis that PRSP expression is important for placental development.

### EXAMPLE 18

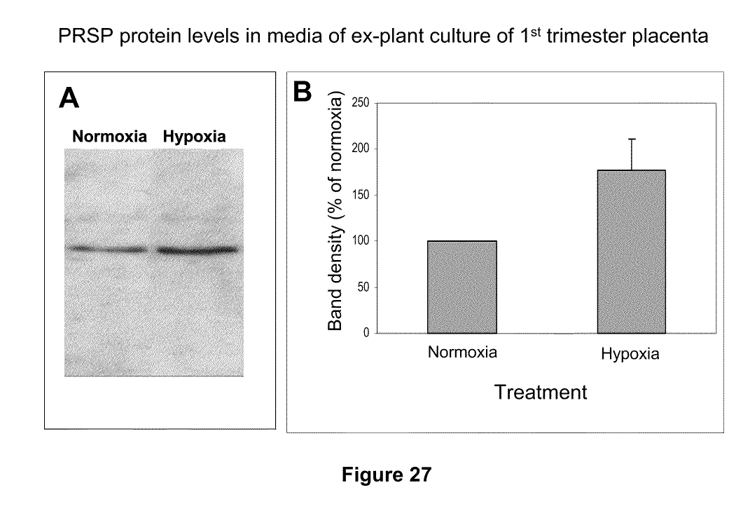

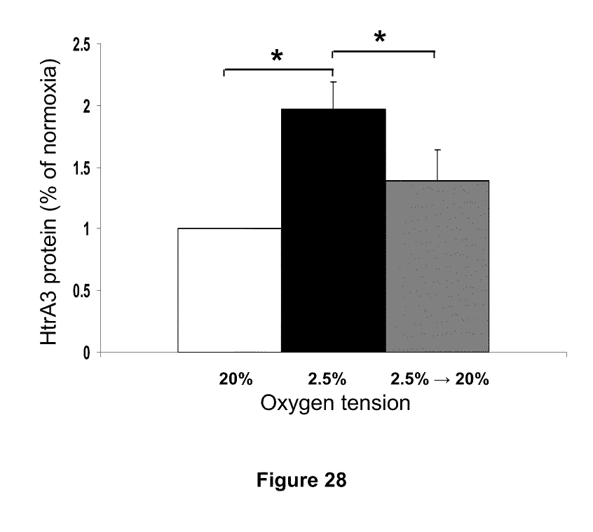

We analyzed the DNA sequence of PRSP gene (SEQ ID NO: 31) and found hypoxia-inducible factor response elements in the promoter, confirming that PRSP is likely regulated by oxygen tension. We then experimentally validated this notion using both human placental explants and syncytial placental cells in culture (FIGS. 27 and 28). We focused on syncytiotrophoblast cells since these produce high amounts of HtrA3 in the 1^{st} trimester placenta and are responsible for placental secretion. Conditioned media from explant culture of 1^{st} trimester placenta (8-10 weeks) was assessed for PRSP. The placental tissues were cultured under normoxic (20% O₂) or hypoxic (5% O₂) conditions, PRSP levels in the media were determined by Western blotting as described above. PRSP levels were much higher in media from the hypoxic culture (FIG. 27), Similarly, syncytialized BeWo cells were grown under normoxic and hypoxic conditions: levels of PRSP in medium was increased by hypoxia and these levels were dramatically reduced when the cells are switched from the hypoxic to normoxic conditions (FIG. 28). Both sets of data demonstrate that PRSP is upregulated by hypoxic conditions.

These data strongly suggest the following: (1) HtrA3 is a marker of oxygen tension in the placenta, (2) HtrA3 reduction at the end of 1^{st} trimester reflects the “oxygen concentration switch” event (thus successful spiral artery de-plugging/remodeling and normal placentation); (3) Abnormally high levels of HtrA3 in early pregnancy (˜13-14 weeks) indicate that the placenta is suffering from abnormal hypoxic environments and has difficulties with vessel remodeling (the root causes of PE).

In summary, our research has provided strong experimental evidence and a solid molecular basis that measuring HtrA3 in maternal blood in early pregnancy may identify pregnancies that are at high risk to develop PE at later stages of pregnancy.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Liang P and Pardee A B

Differential display of eukaryotic messenger RNA by means of the polymerase chain reaction. Science 1992; 257: 967-970.

Liang P and Pardee A B

Distribution and cloning of eukaryotic mRNAs by means of differential display: refinement and optimization. Nucleic Acids Res. 1993; 14: 3269-3275.

Nie G-Y, Li Y, Hampton A L et al.

Identification of monoclonal nonspecific suppressor factor beta (MNSFbeta) as one of the genes differentially expressed at implantation sites compared to interimplantation sites in the mouse uterus. Mol Reprod Dev 2000b; 55: 351-363.

Prowse S

ANZCCART News 2000 13(3):7

1. A method of diagnosing an infertility condition in a human female subject, the method comprising
(a) detecting pregnancy-related serine protease (PRSP) protein in a test sample taken from said subject at between 8 and 20 weeks into pregnancy;
(b) detecting PRSP protein in a control sample from a fertile control human female taken at the same number of weeks into pregnancy in the control as the sample taken from the subject; and
(c) comparing the PRSP protein in the test sample with the PRSP protein detected in the control sample,
in which a change in the PRSP protein in the test sample compared to the control sample is indicative of an infertility condition.

2. The method of claim 1 in which the infertility condition is an inability to achieve or sustain embryo implantation.

3. The method of claim 1 in which the infertility condition is an inability to sustain a normal pregnancy.

4. The method of claim 3 in which the infertility condition is early abortion.

5. The method of claim 1 in which the infertility condition is an insufficiency of placentation.

6. The method of claim 5 in which the infertility condition is pre-eclampsia or IUGR.

7. The method of claim 1 in which the PRSP protein has a sequence selected from the group consisting of the sequences set out in SEQ ID NO: 27, 33, 34 or 39.

8. The method of claim 7 in which the PRSP protein has the sequence set out in SEQ ID NO:33 or SEQ ID NO:34

9. The method of claim 1 in which the PRSP protein is detected using an antibody.

10. The method of claim 9 in which the PRSP protein is detected using an antibody raised against a sequence specific for PRSP.

11. The method of claim 10 in which the PRSP protein is detected using an antibody raised against SEQ ID NO: 52 or 56.

12. The method of claim 9 in which the PRSP protein is detected using an antibody raised against amino acids 133 to 142 or 116 to 126 of SEQ ID NO: 27.

13. The method of claim 1 in which the biological sample is a sample of biological fluid.

14. The method of claim 13 in which the biological fluid is plasma, serum, uterine or bladder washings, urine, saliva or amniotic fluid.

15. The method of claim 1 in which the biological sample is a tissue or cellular sample or extract thereof.

16. The method of claim 15 in which the sample is placental or uterine tissue.

17. The method of claim 1 in which the test sample and the control sample are taken at around 8 weeks.

18. The method of claim 1 in which the test sample and the control sample are taken at around 9 weeks.

19. The method of claim 1 in which the test sample and the control sample are taken at around 10 weeks.

20. The method of claim 1 in which the test sample and the control sample are taken at around 11 weeks.

21. The method of claim 1 in which the test sample and the control sample are taken at around 12 weeks.

22. The method of claim 1 in which the test sample and the control sample are taken at around 13 weeks.

23. The method of claim 1 in which the test sample and the control sample are taken at around 14 weeks.

24. The method of claim 1 in which the test sample and the control sample are taken at around 15 weeks.

25. The method of claim 1 in which the PRSP protein is indicated by a 39 kDa band on Western blot using an antibody raised against SEQ ID NO: 52.

26. The method of claim 25 in which the change in PRSP protein is identified by a decrease in the density of the 39 kDa PRSP band indicative of IUGR.

27. The method of claim 25 in which an increase in the density of the 39 kDa PRSP band is indicative of pre-eclampsia.

28. A method of determining whether a pregnant female is at risk of an infertility condition in a human female subject, the method comprising
(a) detecting pregnancy-related serine protease (PRSP) protein in a test sample taken from said subject in weeks 8-20 of pregnancy;
(b) detecting PRSP protein in a control sample from a fertile control human female taken at the same number of weeks into pregnancy in the control as the sample taken from the subject, or using predetermined control levels of PRSP detected in one or more control sample from one or more fertile control human female; and
(c) comparing the PRSP protein in the test sample with the PRSP protein detected or predetermined in the control sample,
in which a change or significant difference in the PRSP protein in the test sample compared to the control sample is indicative of the risk of an infertility condition.

29. The method of claim 28 in which the infertility condition is an inability to achieve or sustain embryo implantation.

30. The method of claim 28 in which the infertility condition is an inability to sustain a normal pregnancy.

31. The method of claim 30 in which the infertility condition is early abortion.

32. The method of claim 28 in which the infertility condition is an insufficiency of placentation.

33. The method of claim 32 in which the infertility condition is pre-eclampsia or IUGR.

34. The method of claim 28 in which the PRSP protein has a sequence selected from the group consisting of the sequences set out in SEQ ID NO: 33 or 34.

35. The method of claim 28 in which the PRSP protein is detected using an antibody.

36. The method of claim 35 in which the PRSP protein is detected using an antibody raised against SEQ ID NO: 52 or 56.

37. A null mouse in which expression of genes having SEQ ID NO: 26 and or 38 is blocked in which said genes are deleted.

38. An antibody raised against a peptide comprising SEQ ID NO:52 or SEQ ID NO 56.

39. The antibody of claim 38, which is a monoclonal antibody raised against a peptide consisting of SEQ ID NO:52 or SEQ ID NO: 56.

40. (canceled)

41. (canceled)
